(19) United States
(12) Patent Application Publication
(10) Pub. No.: US 2015/0328373 A1
(43) Pub. Date: Nov. 19, 2015

(54) Additives To Increase Degradation Rate Of A Biodegradable Scaffolding And Methods Of Forming Same

(71) Applicant: Abbott Cardiovascular Systems Inc., Santa Clara, CA (US)

(72) Inventors: Stephen D. Pacetti, San Jose, CA (US); Xiao Ma, San Jose, CA (US)

(21) Appl. No.: 14/281,811

(22) Filed: May 19, 2014

(51) Int. Cl. A61L 31/10; B05D 1/18; A61L 31/14

(52) U.S. Cl. CPC A61L 31/10; A61L 31/148; B05D 1/18; A61L 2420/06; A61L 2400/18; A61L 2420/02; B05D 2254/00

(57) ABSTRACT

Methods of making biodegradable polymeric devices, such as stents, with one or more modifications to alter the degradation rate, and the biodegradable polymeric devices are described. Modifications include blending of two polymers, one with a different degradation rate, inclusion of additives to alter the degradation rate, and the use of polymers of a high polydispersity.

### BACKGROUND

1. Field of the Invention

This invention relates to methods of treatment of blood vessels with bioabsorbable polymeric medical devices, in particular, stents.

2. Description of the State of the Art

Until the mid-1980s, the accepted treatment for atherosclerosis, i.e., narrowing of the coronary artery(ies) was by-pass surgery. While effective and evolved to a relatively high degree of safety for such an invasive procedure, by-pass surgery still involves potentially serious complications and in the best of cases an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a relatively short time compared to by-pass surgery and the recovery time was minimal. However, PTCA brought with it other problems such as vasospasm and elastic recoil of the stretched arterial wall which could undo much of what was accomplished and, in addition, it created a new disease, restenosis, the re-clogging of the treated artery due to neointimal hyperplasia.

The next improvement, advanced in the mid-1980s, was the use of a stent to maintain the luminal diameter after PTCA. This for all intents and purposes put an end to vasospasm and elastic recoil but did not entirely resolve the issue of restenosis. That is, prior to the introduction of stents restenosis occurred in about 30-50% of patients undergoing PTCA. Stenting reduced this to about 15-20%, much improved but still more than desirable.

In 2003, drug-eluting stents or DESs were introduced. The drugs initially employed with the DES were cytostatic compounds, that is, compounds that curtailed the proliferation of cells that contributed to restenosis. The occurrence of restenosis was thereby reduced to about 5-7%, a relatively acceptable figure. Thus, stents made from biostable or non-erodible materials, such as metals, have become the standard of care for percutaneous coronary intervention (PCI) as well as in peripheral applications, such as the superficial femoral artery (SFA), since such stents have been shown to be capable of preventing early and later recoil and restenosis.

However, a problem that arose with the advent of DESs was so-called “late stent thrombosis,” the forming of blood clots long after the stent was in place. It was hypothesized that the formation of blood clots was most likely due to delayed healing, a side-effect of the use of cytostatic drugs and durable polymers with suboptimal biocompatibility. One solution is to make a stent from materials that erode or disintegrate through exposure to conditions within the body. Thus, erodible portions of the stent can disappear from the implant region after the treatment is completed, leaving a healed vessel. Stents fabricated from biodegradable, bioabsorbable, and/or bioerodable materials such as bioabsorbable polymers can be designed to completely erode only after the clinical need for them has ended. Like a durable stent, a biodegradable stent must meet time dependent mechanical requirements. For example, it must provide patency for a minimum time period. It is also important for a biodegradable stent to completely degrade from the implant site within a certain period of time.

Thus, there is a continuing need for biodegradable stents that meet both mechanical requirements and degrade once the clinical need for them has ended.

### SUMMARY OF THE INVENTION

Embodiments of the present invention encompass methods of adding additives to polymeric medical devices, such as stents, and the resulting devices, and methods of using the devices.

Some non-limiting embodiments of the invention are described the following numbered paragraphs:

Embodiments of the invention encompass a method of making a stent body for supporting a vascular lumen, the method including at least partially immersing a cylindrical member in a solution comprising a bioabsorbable polymer dissolved in a solvent (a fluid), wherein the bioabsorbable polymer has an inherent viscosity of at least 3.3 dl/g, has a number average molecular weight greater than 250,000 g/mole as measured by GPC using polystyrene standards, or both, and the solution further comprises an additive dissolved, dispersed, or a combination of dissolved and dispersed in the solution; removing the member from the solution, wherein a portion of the solution remains on the surface of the member upon removal from the solution; removing solvent from the solution remaining on the member to form a tubular layer of the bioabsorbable polymer and the additive on the member; optionally, repeating on one or more occasions, the immersion operation, removal from the solution operation, and removal of the solvent operation, to form a final tubular layer of bioabsorbable polymer and the additive on the member of a desired thickness; and forming a stent body from the final tubular layer. The bioabsorbable polymer is poly(L-lactide), a copolymer with L-lactide or L-lactic acid as a constituent monomer, or a combination thereof. In addition, at least one of the following conditions applies:

(a) the additive is the or at least one constituent monomer of the bioabsorbable polymer, and the additive is present at a weight ratio of the additive to the total of the additive and the polymer of about 0.002 to about 0.05;

(b) the additive is an oligomer of the or at least one constituent monomer of the bioabsorbable polymer, and the additive is present at a weight ratio of the additive to the total of the additive and the polymer of about 0.02 to about 0.25;

(c) the additive is a fatty acid at a weight ratio of the additive to the total of the additive and the polymer of about 0.002 to about 0.03;

(d) the additive is a fatty acid ester at a weight ratio of the additive to the total of the additive and the polymer of about 0.002 to about 0.03;

(e) the additive is an unsaturated fatty acid at a weight ratio of the additive to the total of the additive and the polymer of about 0.002 to about 0.03;

(f) the additive is an unsaturated fatty acid ester at a weight ratio of the additive to the total of the additive and the polymer of about 0.002 to about 0.03;

(g) the additive is a hydroxy acid;

(h) the additive is an ester of a hydroxy acid, wherein if the or at least one constituent monomer of the bioabsorbable polymer is a hydroxy acid or a hydroxy acid ester, the additive is a different hydroxy acid ester;

(i) the additive is a dicarboxylic acid;

(j) the additive is an ester of a dicarboxylic acid;

(k) the additive is an anhydride;

(l) the additive is an acid or ester of an acid selected from the group consisting of citric acid; ascorbic acid, erythorbic acid, thiodipropionic acid, cholic acid, desoxycholic acid, glycocholic acid, taurocholic acid, aspartic acid, tartaric acid, glutamic acid, and combinations thereof;

(m) the additive is a metal ion selected from the group consisting of zinc, aluminum, tin, magnesium, calcium, sodium, and iron;

(n) the additive is a hygroscopic additive.

In some embodiments of the invention, such as but not limited to that described in paragraph [0001], the member is removed from the solution in less than 30 seconds.

In some embodiments of the invention, such as but not limited to those described in paragraphs [0001] and [0002], the member is at least partially immersed with its cylindrical axis perpendicular to the surface of the solution.

In some embodiments of the invention, such as but not limited to those described in paragraphs [0001]-[0003], the immersion step is repeated on at least one occasion where the member is rotated 180° prior to repetition of the immersion step.

In some embodiments of the invention, such as but not limited to those described in paragraphs [0001]-[0004], the member is rotated while it is removed from the solution.

In some embodiments of the invention, such as but not limited to those described in paragraphs [0001]-[0005], the method further includes radially expanding the final tubular layer and forming the stent body from the expanded tube.

In some embodiments of the invention, such as but not limited to those described in paragraphs [0001]-[0006], condition (a), (b), or a combination thereof apply, and the additive is selected from the group consisting of D,L-lactide, D,D-lactide, L,L-lactide, meso-lactide, glycolide, caprolactone, trimethylene carbonate, p-dioxanone, γ-valeroactone, γ-undecalactone, β-methyl-δ-valerolactone, anhydrides, orthocarbonates, phosphazenes, orthoesters, amino acids, and combinations thereof

In some embodiments of the invention, such as but not limited to those described in paragraphs [0001]-[0007], condition (c), (d), or a combination thereof apply, and the fatty acid, the fatty acid of the fatty acid ester, or a combination thereof is selected from the group consisting acetic acid, propanoic acid, butyric acid, caprylic acid, caproic acid, capric acid, lauric acid, myristic acid, palmitic acid, stearic acid, arachidic acid, behenic acid, lignoceric acid, cerotic acid, and combinations thereof.

In some embodiments of the invention, such as but not limited to those described in paragraphs [0001]-[0008], condition (e), (f), or a combination thereof apply, and the unsaturated fatty acid, the unsaturated fatty acid of the unsaturated fatty acid ester, or a combination thereof, is selected from the group consisting of myristoleic acid, palmitoleic acid, spienic acid, oleic acid, elaidic acid, vaccenic acid, linoleic acid, linoelaidic acid, alpha-linolenic acid, arachidonic acid, eicosapentaenoic acid, erucic acid, and docosahexaenoic acid, and combinations thereof.

In some embodiments of the invention, such as but not limited to those described in paragraphs [0001]-[0009], condition (g), (h), or a combination thereof apply, and the hydroxy acids are selected from the group consisting of L-lactic acid, D-lactic acid, glycolic acid, 2-hydroxybutyric acid, 3-hydroxybutyric acid, 2-hydroxyvaleric acid, 3-hydroxybutyric acid, 4-hydroxybutyric acid, 3-hydroxyvaleric acid, 4-hydroxyvaleric acid, 5-hydroxyvaleric acid, dimethylglycolic acid, β-hydroxypropanic acid, α-hydroxybutyric acid, α-hydroxycaproic acid, β-hydroxycaproic acid, γ-hydroxycaproic acid, δ-hydroxycaproic acid, δ-hydroxymethylcaproic acid, ε-hydroxycaproic acid, ε-hydroxymethylcaproic acid, citric acid, tartaric acid, and combinations thereof.

In some embodiments of the invention, such as but not limited to those described in paragraphs [0001]-[0010], condition (i), (j), or a combination thereof apply, and the dicarboxylic acid, the dicarboxylic acid of the ester, or a combination thereof, is selected from the group consisting of oxalic acid, malonic acid, succinic acid, glutaric acid, adipic acid, pimelic acid, suberic acid, azelaic acid, sebacic acid, orthophthalic acid, isophthalic acid, terephthalic acid, and combinations thereof.

In some embodiments of the invention, such as but not limited to those described in paragraphs [0001]-[0011], condition (k) applies, and the anhydride is selected from the group consisting of succinic anhydride, glutaric anhydride, maleic anhydride, acetic anhydride, propanoic anhydride, butyric anhydride, valeric anhydride, caproic anhydride, heptanoic anhydride, phthalic anhydride, and benzoic anhydride, and combinations thereof.

In some embodiments of the invention, such as but not limited to those described in paragraphs [0001]-[0012], condition (1) applies.

In some embodiments of the invention, such as but not limited to those described in paragraphs [0001]-[0013], condition (m) applies.

In some embodiments of the invention, such as but not limited to those described in paragraphs [0001]-[0014], condition (n) applies, and the hygroscopic additive is selected from the group consisting of sodium phosphate, sodium biphosphate, sodium pyrophosphate, potassium phosphate, sodium carbonate, sodium bicarbonate, potassium carbonate, sodium sulfate, magnesium sulfate, sodium chloride, potassium chloride, calcium ascorbate, calcium propionate, calcium sorbate, calcium carbonate, calcium citrate, calcium glycerophosphate, calcium oxide, calcium pantothenate, calcium phosphate, calcium pyrophosphate, calcium sulfate, calcium chloride, calcium gluconate, calcium hydroxide, calcium lactate, calcium oxide, magnesium chloride, methyl cellulose, ethyl cellulose, sodium carboxymethylcellulose, cellulose acetate, and combinations thereof

In some embodiments of the invention, such as but not limited to those described in paragraphs [0001]-[0015], condition (n) applies, and the hygroscopic additive is present at a weight ratio of the additive to the total of the additive and the polymer of about 0.002 to about 0.05; and wherein the additive is propylene glycol, glycerol, or a combination thereof.

In some embodiments of the invention, such as but not limited to those described in paragraphs [0001]-[0016], if the bioabsorbable polymer is poly(L-lactide), the additive is other than L-lactide.

Embodiments of the invention encompass a method of making a stent body for supporting a vascular lumen, including at least partially immersing a cylindrical member in a solution comprising a bioabsorbable polymer dissolved in a solvent (a fluid), wherein the bioabsorbable polymer has an inherent viscosity of at least 3.3 dl/g, has a number average molecular weight greater than 250,000 g/mole as measured by GPC using polystyrene standards, or both; removing the member from the solution, wherein a portion of the solution remains on the surface of the member upon removal from the solution; removing solvent from the solution remaining on the member to form a tubular layer of the bioabsorbable polymer on the member; optionally, repeating on one or more occasions the immersion operation, removal from the solution operation, and removal of the solvent operation to form a final tubular layer of bioabsorbable polymer on the member of a desired thickness; and forming a stent body from the final tubular layer. The bioabsorbable polymer is poly(L-lactide), a copolymer with L-lactide or L-lactic acid as a constituent monomer, or a combination thereof.

Additionally, at least one of the following conditions applies:

(a) the polydispersity of the bioabsorbable polymer is at least 4 or greater than 4;

(b) the solution further comprises a second bioabsorbable polymer, the second bioabsorbable polymer being poly(glycolide), a copolymer where one constituent monomer is glycolide, poly(D,L-lactide), a polymer where the constituent monomers are D-lactide and L-lactide, dioxanone, 4-hydroxybutyrate, and trimethylene carbonate, a copolymer where the constituent monomers are D-lactide, and at least one member of the group of dioxanone, 4-hydroxybutyrate, and trimethylene carbonate, a copolymer where the constituent monomers are L-lactide, and at least one member of the group of dioxanone, 4-hydroxybutyrate, and trimethylene carbonate, a copolymer where the constituent monomers are D-lactide and L-lactide, and at least one member of the group of dioxanone, 4-hydroxybutyrate, and trimethylene carbonate, a copolymer where at least one constituent monomer is a member of the group of dioxanone, 4-hydroxybutyrate, and trimethylene carbonate, or a combination thereof.

In some embodiments of the invention, such as but not limited to that described in paragraph [0018], condition (a) applies.

In some embodiments of the invention, such as but not limited to those described in paragraphs [0018] and [0019], condition (b) applies.

In some embodiments of the invention, such as but not limited to those described in paragraph [0020], the second bioabsorbable polymer is of a number average molecular weight of not more than one fifth of the number average molecular weight of the first polymer.

In some embodiments of the invention, such as but not limited to that described in paragraphs [0020] and [0021], the second bioabsorbable polymer is a copolymer where one constituent monomer is glycolide selected from the group consisting of poly(L-lactide-co-glycolide), poly(D-lactide-co-glycolide), poly(D,L-lactide-co-glycolide), poly(glycolide-co-dioxanone), poly(glycolide-co-4-hydroxybutyrate), poly(glycolide-co-caprolactone), poly(glycolide-co-trimethylene carbonate), and combinations thereof.

In some embodiments of the invention, such as but not limited to those described in paragraphs [0001]-[0022], the member is totally immersed during at least one immersion operation.

Embodiments of the invention encompass polymer scaffold including a device body made of a bioabsorbable polymer, and optionally, an additive; and at least one of the following conditions applies:

(a) the polydispersity of the bioabsorbable polymer is at least 4 or greater than 4;

(b) the bioabsorbable polymer is poly(L-lactide), a copolymer where one constituent monomer is L-lactide, or a combination thereof; and a second bioabsorbable polymer is blended with the bioabsorbable polymer, the second bioabsorbable polymer being poly(glycolide), a copolymer where one constituent monomer is glycolide, poly(D,L-lactide), a polymer where the constituent monomers are D-lactide and L-lactide, dioxanone, poly(4-hydroxybutyrate), and poly(trimethylene carbonate), a copolymer where the constituent monomers are D-lactide, and at least one member of the group of polydioxanone, poly(4-hydroxybutyrate), and poly(trimethylene carbonate), a copolymer where the constituent monomers are L-lactide, and at least one member of the group of polydioxanone, poly(4-hydroxybutyrate), and poly(trimethylene carbonate), a copolymer where the constituent monomers are D-lactide and L-lactide, and at least one member of the group of polydioxanone, poly(4-hydroxybutyrate), and poly(trimethylene carbonate), a copolymer where at least one constituent monomer is a member of the group of polydioxanone, poly(4-hydroxybutyrate), and poly(trimethylene carbonate), or a combination thereof;

(c) an additive is present, and if the additive is the or at least one constituent monomer of the bioabsorbable polymer, the additive is present at a weight ratio of the additive to the total of the additive and the polymer of about 0.002 to about 0.05; if the additive is an oligomer of the or at least one constituent monomer of the bioabsorbable polymer, the additive is present at a weight ratio of the additive to the total of the additive and the polymer of about 0.02 to about 0.25; if the additive is a fatty acid, a fatty acid ester, an unsaturated fatty acid, an unsaturated fatty acid ester, the additive is present at a weight ratio of the additive to the total of the additive and the polymer of about 0.002 to about 0.03.

In some embodiments of the invention, such as but not limited to that described in paragraph [0024], the bioabsorbable polymer has an inherent viscosity of at least 3.3 dl/g, has a number average molecular weight greater than 250,000 g/mole as measured by GPC using polystyrene standards, or both.

In some embodiments of the invention, such as but not limited to those described in paragraphs [0024] and [0025], condition (a) applies, and the bioabsorbable polymer is poly(L-lactide), a copolymer where one constituent monomer is L-lactide, or a combination thereof

In some embodiments of the invention, such as but not limited to those described in paragraphs [0024]-[0026], condition (b) applies.

In some embodiments of the invention, such as but not limited to those described in paragraph [0027], the second polymer is of a number average molecular weight of not more than one fifth of the number average molecular weight of the first polymer.

In some embodiments of the invention, such as but not limited to that described in paragraphs [0027] and [0028], the second bioabsorbable polymer is a copolymer where one constituent monomer is glycolide selected from the group consisting of poly(L-lactide-co-glycolide), poly(D-lactide-co-glycolide), poly(D,L-lactide-co-glycolide), poly(glycolide-co-caprolactone), poly(glycolide-co-dioxanone), poly(glycolide-co-4-hydroxybutyrate), poly(glycolide-co-trimethylene carbonate), and combinations thereof.

In some embodiments of the invention, such as but not limited to those described in paragraphs [0024]-[0028], condition (c) applies, and the additive is a member of at least one of the following groups:

(a) the constituent monomer(s) of the bioabsorbable polymer;

(b) oligomers formed from the or at least one constituent monomer of the bioabsorbable polymer;

(c) fatty acids;

(d) fatty acid esters;

(e) unsaturated fatty acids;

(f) unsaturated fatty acid esters;

(g) hydroxy acids;

(h) esters of hydroxy acids, wherein if the or at least one constituent monomer of the bioabsorbable polymer is a hydroxy acid or hydroxyacid diester including cyclic diesters, the additive is a different hydroxy acid;

(i) dicarboxylic acids;

(j) esters of dicarboxylic acids;

(k) anhydrides;

(l) acids, esters of an acid, and combinations thereof, wherein the acid is selected from the group consisting of an acid or ester of an acid selected from the group consisting of citric acid; ascorbic acid, erythorbic acid, thiodipropionic acid, cholic acid, desoxycholic acid, glycocholic acid, taurocholic acid, aspartic acid, tartaric acid, glutamic acid, and combinations thereof;

(m) metal ions selected from the group consisting of zinc, iron, tin, magnesium, calcium, sodium and aluminum;

(n) the additive is a hygroscopic additive.

In some embodiments of the invention, such as but not limited to those described in paragraph [0029], the bioabsorbable polymer is poly(L-lactide), a copolymer where one constituent monomer is L-lactide, or a combination thereof.

In some embodiments of the invention, such as but not limited to that described in paragraph [0030], the bioabsorbable polymer is poly(L-lactide), and the additive is other than L-lactide.

In some embodiments of the invention, such as but not limited to those described in paragraphs [0001]-[0031], the bioabsorbable polymer has an inherent viscosity of at least 4.0 dl/g, at least 4.5 dl/g, at least 5.0 dl/g, at least 6.0 dl/g, at least 7.0 dl/g, or at least 8.0 dl/g in chloroform at 25° C., but not more than 25 dl/g in chloroform at 25° C.

In some embodiments of the invention, such as but not limited to those described in paragraphs [0001]-[0032], the bioabsorbable polymer has a number average molecular weight greater than 275,000 g/mole, greater than 300,000 g/mole, greater than 350,000 g/mole, greater than 400,000 g/mole, greater than 500,000 g/mole, greater than 600,000 g/mole, or greater than 750,000 g/mole, but not greater than 2,500,000 g/mole.

In some embodiments of the invention, such as but not limited to those described in paragraphs [0001]-[0033], the bioabsorbable polymer has a weight average molecular weight greater than 300,000 g/mole, greater than 350,000 g/mole, greater than 400,000 g/mole, greater than 450,000 g/mole, greater than 500,000 g/mole, greater than 675,000 g/mole, or greater than 800,000 g/mole, but not greater than 3,000,000 g/mole.

### BRIEF DESCRIPTION OF THE DRAWINGS

FIG. 1 depicts an exemplary stent.

FIG. 2 depicts a cross-section of tube of multiple layers, respectively.

FIGS. 3A-C illustrate the dip coating process of the present invention.

FIGS. 4A and 4B depict radial and axial cross-sections, respectively, of a coated mandrel.

FIG. 5 depicts a mandrel mounting disk having a plurality of holes configured to hold mandrels for a dip coating operation.

FIG. 6A depicts a system for controlled dip coating of mandrels mounted on the mounting disk of FIG. 5.

FIG. 6B shows the system of FIG. 6A with the mounting disk and mounted mandrels removed from a solution.

### DETAILED DESCRIPTION OF THE INVENTION

Use of the term “herein” encompasses the specification, the abstract, and the claims of the present application.

Use of the singular herein includes the plural and vice versa unless expressly stated to be otherwise. That is, “a” and “the” refer to one or more of whatever the word modifies. For example, “a drug” may refer to one drug, two drugs, etc. Likewise, “the stent” may refer to one, two or more stents and “the polymer” may mean one polymer or a plurality of polymers. By the same token, words such as, without limitation, “stents” and “polymers” would refer to one stent or polymer as well as to a plurality of stents or polymers unless it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. With the preceding discussion in mind, a numerical value herein that is modified by a word of approximation may vary from the stated value by ±15% in some embodiments, by ±10% in some embodiments, by ±5% in some embodiments, or in some embodiments, may be within the 95% confidence interval.

As used herein, any ranges presented are inclusive of the end-points. For example, “a temperature between 10° C. and 30° C.” or “a temperature from 10° C. to 30° C.” includes 10° C. and 30° C., as well as any temperature in between. In addition, throughout this disclosure, various aspects of this invention may be presented in a range format. The description in range format is merely for convenience and brevity and should not be construed as an inflexible limitation on the scope of the invention. Accordingly, the description of a range should be considered to have specifically disclosed all the possible subranges as well as individual numerical values, both integers and fractions, within that range. As an example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. Unless expressly indicated, or from the context clearly limited to integers, a description of a range such as from 1 to 6 should be considered to have specifically disclosed subranges 1.5 to 5.5, etc., and individual values such as 3.25, etc. This applies regardless of the breadth of the range.

Embodiments of the present invention are applicable to treatment of coronary and peripheral disease in coronary arteries and various peripheral vessels. Embodiments of the present invention encompass implantation in the cerebral, carotid, coronary, aortic, renal, iliac, renal, femoral, popliteal, and tibial vasculature. Coronary arteries refer generally to arteries that branch off the aorta to supply the heart muscle with oxygenated blood. Peripheral arteries refer generally to blood vessels outside the heart and brain.

In both coronary artery disease and peripheral artery disease, the arteries become hardened and narrowed or stenotic and restrict blood flow. In the case of the coronary arteries, blood flow is restricted to the heart, while in the peripheral arteries blood flow is restricted leading to the brain, kidneys, stomach, arms, legs, and feet. The narrowing is caused by the buildup of cholesterol and other material, called plaque, on the inner walls of the vessel. Such narrowed or stenotic portions are often referred to as lesions. Artery disease also includes the reoccurrence of stenosis or restenosis that occurs after an angioplasty treatment. Although there are probably several mechanisms that lead to restenosis of arteries, an important one is the inflammatory response, which induces tissue proliferation around an angioplasty site. The inflammatory response can be caused by the balloon expansion used to open the vessel, or if a stent is placed, by the foreign material of the stent itself.

As noted above, embodiments of the present invention are applicable to treatment of coronary and peripheral disease in coronary arteries and various peripheral vessels including the superficial femoral artery, the iliac artery, and carotid artery. The embodiments are further applicable to various types of medical devices, such as stents, and various stent types, such as self-expandable and balloon expandable stents.

A stent or scaffold is a type of implantable medical device. As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, stent-expandable stents, stent-grafts, grafts, artificial heart valves, foramen ovale closure devices, cerebrospinal fluid shunts, orthopedic fixation devices, and intrauterine devices.

Other medical devices may be referred to as insertable medical devices, that are any type of appliance that is totally or partly introduced, surgically or medically, into a patient's body or by medical intervention into a natural orifice, but the device does not remain in the patient's body after the procedure.

As noted above a stent is a type of implantable medical device. Stents are generally cylindrically shaped and function to hold open, and sometimes expand, a segment of a blood vessel or other vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease. A stent can be used in, without limitation, the neuro, carotid, coronary, pulmonary, aorta, renal, biliary, iliac, femoral and popliteal, as well as other peripheral vasculatures, and in other bodily lumens such as the urethra or bile duct. A stent can be used in the treatment or prevention of disorders such as, without limitation, atherosclerosis, vulnerable plaque, thrombosis, restenosis, hemorrhage, vascular dissection and perforation, vascular aneurysm, chronic total occlusion, claudication, anastomotic proliferation, bile duct obstruction and ureter obstruction.

Another type of medical device is a vascular catheter, a type of insertable device. A vascular catheter is a thin, flexible tube with a manipulating means at one end, referred to as the proximal end, which remains outside the patient's body, and an operative device at or near the other end, called the distal end, which is inserted into the patient's artery or vein. The catheter may be introduced into a patient's vasculature at a point remote from the target site, e.g., into the femoral artery of the leg where the target is in the vicinity of the heart. The catheter is steered, assisted by a guide wire than extends through a lumen, which is a passageway or cavity, in the flexible tube, to the target site whereupon the guide wire is withdrawn. After the guidewire is withdrawn, the lumen may be used for the introduction of fluids, often containing drugs, to the target site. For some vascular catheters there are multiple lumens allowing for the passage of fluids without removal of the guidewire. A catheter may also be used to deliver a stent or may be used to deliver a balloon used in angioplasty.

As used herein, a “balloon” refers to the well-known in the art device, usually associated with a vascular catheter, that comprises a relatively thin, flexible material, forming a tubular membrane, that when positioned at a particular location in a patient's vessel may be expanded or inflated to an outside diameter that is essentially the same as the inside or luminal diameter of the vessel in which it is placed. In angioplasty procedures, the balloon is expanded to a size larger than the luminal diameter of the vessel, as it is a diseased state, and closer to the luminal size of a healthy reference section of vessel. In addition to diameter, a balloon has other dimensions suitable for the vessel in which it is to be expanded. Balloons may be inflated, without limitation, using a liquid medium such as water, aqueous contrast solution, or normal saline solution, that is, saline that is essentially isotonic with blood.

A “balloon catheter” refers to medical device which is a system of a catheter with a balloon at the end of the catheter.

A balloon, a catheter, and a stent differ. Stents are typically delivered to a treatment site by being compressed or crimped onto a catheter or onto a catheter balloon, and then delivered through narrow vessels to a treatment site where the stent is deployed. Deployment involves expanding the stent to a larger diameter, typically to the diameter of the vessel, once it is at the treatment site. Stents can be self-expanding or balloon expandable. The expanded stent is capable of supporting a bodily lumen for an extended period of time. In contrast, a balloon has a wall thickness that is so thin that the tubular membrane cannot support a load at a given diameter unless inflated with a fluid, such as a liquid or gas. Furthermore, a balloon is a transitory device that is inserted in the patient's body for only a limited time for the purpose of performing a specific procedure or function. Unlike a stent, dilatation balloons are not permanently implanted within the body. Moreover, vascular catheters have a length to diameter ratio of at least 50/1.

The structure of a stent is typically a generally cylindrical or tubular form (but the precise shape may vary from the shape of a perfect cylinder), and the tube or hollow cylinder may be perforated with passages that are slots, ovoid, circular, similar shapes, or any combination thereof. In some embodiments, the perforations form at least 10%, preferably at least 20%, and more preferably at least 25%, and in some embodiments, at least 30%, but not more than 99% of the exterior surface area of the tube. A stent may be composed of scaffolding that includes a pattern or network of interconnecting structural elements or struts. The scaffolding can be formed from tubes, or sheets of material, which may be perforated or unperforated, rolled into a cylindrical shape and welded or otherwise joined together to form a tube. A pattern may be formed in the tube by laser cutting, chemical etching, etc.

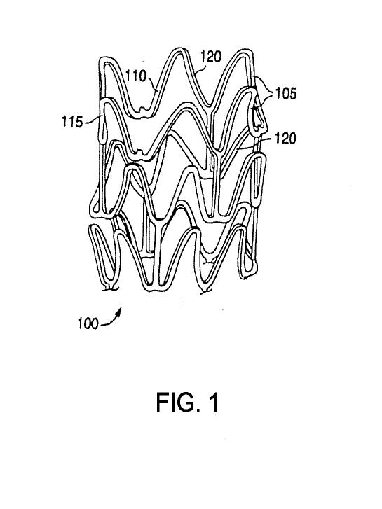

An example of a stent 100 is depicted in FIG. 1. As noted above, a stent may be a scaffolding having a pattern or network of interconnecting structural elements or struts 105, which are designed to contact the luminal walls of a vessel and to maintain vascular patency, that is to support the bodily lumen. Struts 105 of stent 100 include luminal faces or surfaces 110 (facing the lumen), abluminal faces or surfaces 115 (facing the tissue), and side-wall faces or surfaces 120. The pattern of structural elements 105 can take on a variety of patterns, and the structural pattern of the device can be of virtually any design. Typical expanded diameters of a stent range from approximately 1.5 mm to 35 mm, preferably from approximately 2 mm to 10 mm, and for a coronary stent, from 1.5-6.0 mm. The length to diameter ratio of a stent is typically from 2 to 25. The embodiments disclosed herein are not limited to stents, or to the stent pattern, illustrated in FIG. 1.

Other types of stents are those formed of wires, such as the Wallsten stent, U.S. Pat. No. 4,655,771, and those described in U.S. Pat. No. 7,018,401 B1 and U.S. Pat. No. 8,414,635 B2. Those described in U.S. Pat. No. 7,018,401 B1 and U.S. Pat. No. 8,414,635 B include, but are not limited to, a plurality of shape memory wires woven together to form a body suitable for implantation into an anatomical structure. These devices may be of a substantially uniform diameter, or may have a variable diameter such as an hourglass shape. Other stent forms include helical coils.

The body, scaffolding, or substrate of a stent may be primarily responsible for providing mechanical support to walls of a bodily lumen once the stent is deployed therein. The “device body” of a medical device may be the functional device without a coating or layer of material different from that of which the device body is manufactured has been applied. If a device is a multi-layer structure, the device body may be the layer(s) that form the functional device, and for a stent this would be the layer(s) which support the bodily lumen. “Outer surface” refers to any surface however spatially oriented that is in contact, or may be in contact, with bodily tissue or fluids. A stent body, scaffolding, or substrate can refer to a stent structure formed by laser cutting a pattern into a tube or a sheet that has been rolled into a cylindrical shape with or without subsequent processing such as cutting, to a wire or woven mesh, or to a helical coil.

Implantable and insertable medical devices can be made of virtually any material including metals and/or polymers including both bioabsorbable polymers, biostable polymers, and combinations thereof.

Obviously, a stent formed of a biostable or durable material would remain in the body until removed. There are certain disadvantages to the presence of a permanent implant in a vessel such as compliance mismatch between the stent and vessel and risk of embolic events. The presence of a stent may affect healing of a diseased blood vessel. To alleviate such disadvantages, stent can be made from materials that erode or disintegrate through exposure to conditions within the body. Thus, erodible portions of the stent can disappear from the implant region after the treatment is completed, leaving a healed vessel. Stents fabricated from biodegradable, bioabsorbable, and/or bioerodable materials such as bioabsorbable polymers can be designed to completely erode only after the clinical need for them has ended.

As noted above, the embodiments of the present invention encompass devices that are bioabsorbable. As used herein, the terms “biodegradable,” “bioabsorbable,” “bioresorbable,” and “bioerodable” are used interchangeably and refer to materials, such as but not limited to, polymers, which are capable of being completely degraded and/or eroded when exposed to bodily fluids such as blood and can be gradually resorbed, absorbed, and/or eliminated by the body. The processes of breaking down and absorption of the polymer can be caused by, for example, hydrolysis and metabolic processes. Conversely, the term “biostable” refers to materials that are not biodegradable.

The prevailing mechanism of degradation of biodegradable polymers is chemical hydrolysis of the hydrolytically unstable backbone. In a bulk eroding polymer, polymer is chemically degraded and material is lost from the entire polymer volume in a spatially uniform manner. As the polymer degrades, the molecular weight decreases. The reduction in molecular weight is followed by a reduction in mechanical properties, and then erosion or mass loss. The decrease in mechanical properties eventually results in loss of mechanical integrity demonstrated by fragmentation of the device. Phagocytic action and metabolization of the fragments occurs, resulting in a rapid loss of polymer mass.

The treatment of artery disease with a stent of the present invention has time dependent properties once it is implanted which enable the treatment and healing of a diseased section of the vessel. In particular, the molecular weight, the mechanical properties, the mechanical integrity, and mass change with time. After deployment at a diseased section artery, the stent supports the section at an increased diameter for a period of time. Due to a decrease in molecular weight, the radial strength degrades to the point that the stent can no longer support the walls of the section of the vessel. “Radial strength” of a stent is defined as the pressure at which a stent experiences irrecoverable deformation. The loss of radial strength is followed by a gradual decline of mechanical integrity.

Mechanical integrity refers to the size, shape, and connectivity of the structural elements of the stent. For example, the shape refers to the generally tubular shape of the stent. This tubular shape may be formed by the cylindrically-shaped rings connected by the linking elements of the pattern. Mechanical integrity starts to be lost when fractures appear or propagate in structural elements of the stent due to chemical degradation (molecular weight decline). Further loss of mechanical integrity occurs when there is breaking or loss of connectivity in structural elements.

The initial clinical need for any stent is to provide mechanical support to maintain patency or keep a vessel open at or near the deployment diameter. The patency provided by the stent allows the stented segment of the vessel to undergo positive remodeling at the increased deployed diameter. By maintaining the patency of the stented segment at this stage, the stent prevents negative remodeling. Remodeling refers generally to structural changes in the vessel wall that enhance its load-bearing ability so that the vessel wall in the stented section can maintain an increased diameter in the absence of the stent support, the restoration of normal anatomy, and ultimately, normal function of the vessel. A period of patency is required in order to obtain permanent positive remodeling.

During this time period, the stent inhibits or prevents the natural pulsatile function of the vessel. The stent structure prevents recoil and maintains a circular lumen while the vessel remodels and molds itself to the stented diameter, which corresponds to positive remodeling. Early recoil before sufficient modeling takes place can result in negative remodeling, referring to molding of the stent to a diameter significantly less than the original stented diameter, for example, 50% or less than the original deployment diameter.

As the polymer of the stent degrades, the radial strength of the stent decreases and the load of the vessel is gradually transferred from the stent to the remodeled vessel wall. Remodeling of the vessel wall continues after loss of radial strength of the stent. Before the stent loses mechanical integrity, it is desirable for the stent structural elements to become incorporated in the vessel wall by a neointimal layer with endothelium. The stent then becomes discontinuous which allows vasomotion. The vessel wall continues to remodel as the vessel moves due to vasomotion. The stent eventually erodes away completely leaving a healed vessel with an increased diameter and which can exhibit vasomotion the same or similar to a healthy vessel section. In contrast, a biostable stent would not get to the point of allowing for vasomotion as the stent inhibits or prevents the natural pulsatile function of the vessel.

A stent has certain mechanical requirements such as high radial strength, high modulus, high fracture toughness, and high fatigue resistance including bending fatigue for endovascular applications. A stent that meets such requirements greatly facilitates the delivery, deployment, and treatment of a diseased vessel. With respect to radial strength, the strength to weight ratio of polymers is usually smaller than that of metals. A polymeric stent with inadequate mechanical properties can result in mechanical failure, strut fracture, or recoil inward after implantation into a vessel. To compensate for the lower strength to weight ratio of polymers, a polymeric stent can require significantly thicker struts than a metallic stent, which results in an undesirably large profile.

Other ways to compensate for the lower strength to weight ratio of polymers (as compared to metals) is the use of processing which increases the strength and fracture toughness of the final stent product. The strength and fracture toughness may be increased by induced biaxial orientation of polymers in the hoop or circumferential and/or axial direction, a particular range of the degree of crystallinity, and small dispersed crystallites. As an example, a stent may be made from an extruded polymer tube that has been radially expanded and axially stretched to provide the induced orientation. The polymer tube may be expanded by blow molding with a percent radial expansion between 200% and 500%, and a percent axial stretch from 20% to 200%. In some embodiments, the extruded polymer tubing may have a percent of axial stretch from 100% to 400%. Additionally, the blow molding process may be performed in a manner that results in small crystallites dispersed through an amorphous matrix that enhances fracture toughness. The degree of crystallinity may be controlled. The stent may be formed from the expanded tube by laser cutting the tubing in its expanded state.

However, such processing typically involves higher temperatures and subjects the polymer to shear forces both of which induce degradation of many polymers. In particular, polyesters, such as without limitation, poly(L-lactide), are subject to degradation at elevated temperatures. In addition, it is known that radiation sterilization can further reduce the molecular weight of most bioresorbable polymers.

Another method which may be used in addition to or instead of the other described methods is to use polymers of high molecular weight which have desirable mechanical properties. Polymers of high molecular weight may take longer to degrade as the time for total mass loss is a function of initial molecular weight. In addition, high molecular weight polymers are difficult, if not impossible, to melt process.

As noted above, a biodegradable stent must meet time dependent mechanical requirements such as providing patency for a minimum time period. However, it is also important for a biodegradable stent to completely degrade from the implant site within a certain period of time. In addition, the requisite or desired degradation time varies between types of applications, i.e. coronary or peripheral. For coronary and peripheral applications, it is believed that the mechanical integrity should remain intact for at least 3 to 6 months without severe fractures (e.g., breaking of multiple struts with formation of fragments) after implantation to allow incorporation of stent into vessel wall. Additionally, it is believed that radial strength should be maintained for at least about 3 months to prevent negative remodeling. The radial strength is expected to be lost prior to the mechanical integrity and the start of the loss of mechanical integrity is expected to start before mass loss.

Various embodiments of the present invention encompass an implantable device, such as a stent, having a device body or scaffolding formed or fabricated from a bioabsorbable polymer having a high molecular weight, but modified such that degradation behavior appropriate to the application of the stent. Various embodiments of the invention encompass solvent based methods of forming medical devices, such as stents, having a device body or scaffolding formed or fabricated from a bioabsorbable polymer having a high molecular weight, but modified such that the degradation behavior is appropriate to each application of the stent. The modifications of the polymer degradation rate to be subsequently discussed may be used individually, or in combination.

Although the discussion that follows may make reference to a stent or stents as the medical device, the embodiments of the present invention are not so limited, and encompass any medical device which may benefit from the embodiments of the invention.

As used herein, “polymeric stent” refers to a stent having a scaffolding that is made completely, or substantially completely, from a polymer, or the scaffolding is made from a composition including a polymer and a material. If the scaffolding is made from a composition including a polymer and a material, the polymer is a continuous phase of the scaffolding, the scaffolding is at least 50% by weight polymer, or the scaffolding is at least 50% by volume polymer. In some embodiments, a polymeric stent may have a scaffolding made from a composition including a polymer and a material that is at least 70%, at least 80%, at least 90%, or at least 95% by volume or by weight polymer. Analogous definitions apply to a polymeric tube, or a polymeric medical device except that the reference to the scaffolding would be replaced by “tube” for a polymer tube and “device body” for a medical device.

Exemplary of semicrystalline polymers that may be used individually, or in combination, as the bioabsorbable polymer in embodiments of the present invention include, without limitation, poly(L-lactide) (PLLA), polyglycolide (PGA), polymandelide (PM), polycaprolactone (PCL), poly(trimethylene carbonate) (PTMC), polydioxanone (PDO), poly(4-hydroxy butyrate) (PHB), and poly(butylene succinate) (PBS). A non-limiting exemplary amorphous polymer that may be used as the bioabsorbable polymer in the embodiments of the present invention is poly(DL-lactide) (PDLLA). Additionally, block, random, and alternating copolymers of the above polymers may also be used in embodiments of the present invention, for example, poly(L-lactide-co-glycolide).

In preferred embodiments, the polymer is Poly(L-lactide) (PLLA), a polymer with L-lactide or L-lactic acid as a constituent monomer of at least 30 mol %, preferably, at least 50 mol %, more preferably 60 mol %, and even more preferably at least 70 mol %, and up to 98 mol %, or a combination thereof. In some embodiments, the polymer is a poly(D,L-lactide-co-L-lactide) polymer of about 1-10 mol %, such as 4 mol % D,L-lactide, and 99-90 mol %, such as 96 mol %, L-lactide, where mol % is % in terms of moles. Poly(L-lactide) is attractive as a stent material due to its relatively high strength and a rigidity at human body temperature, about 37° C. The glass transition temperature (Tg) of PLLA varies between approximately 50 to 80° C., or more narrowly between 55 and 65° C., depending on crystallinity, microstructure, and molecular weight. Since typically, PLLA has glass transition temperature between about 60 and 65° C. (Medical Plastics and Biomaterials Magazine, March 1998), it remains stiff and rigid at human body temperature. This property facilitates the ability of a stent to maintain a lumen at or near a deployed diameter without significant recoil.

PLLA has an in vitro degradation time of up to 3 years (Medical Plastics and Biomaterials Magazine, March 1998; Medical Device Manufacturing & Technology 2005). The degradation time is the time required for complete loss of mass of a polymer construct, such as a stent. The degradation time in vivo is shorter and depends on the implant location and animal model. In addition to an erosion profile, a PLLA stent has associated molecular weight and mechanical property (e.g., strength) profiles.

The degradation behavior of a stent made from a semicrystalline degradable polyester, such as PLLA, is a complex function of several properties of the material and stent body. These properties include the intrinsic hydrolysis rate of the polymer (i.e., the chain scission reactions of the polymer backbone), the degree of crystallinity, the morphology (size and distribution of crystallite domains in the amorphous matrix), molecular weight (as measured by the inherent viscosity, number, weight, or viscosity average molecular weight), and stent body parameters (pattern, strut dimensions and the surface to volume ratio).

With respect to hydrolytic degradation, a hydrolytic degradation model for aliphatic polyesters having the form Mn(t)=Mn(0)exp(−Kt), wherein Mn(t) is the number average molecular weight at time t, Mn(0) is the number average molecular weight at t=0, and K is the hydrolytic degradation rate constant. Pitt, C. G., J. of Applied Polymer Science: 26, 3779-3787 (1981); Pitt, C. G., Biomaterials: 2, 215-220 (1981); Weir, N. A., Proceedings of the Institution of Mechanical Engineers, Part H: J. of Engineering in Medicine: 218, 307-319 (2004); Weir, N. A., Part H: J. of Engineering in Medicine 218, 321-330 (2004). The assumptions inherent in the model are reasonable provided that the mass loss has not occurred, since mass loss would affect the concentrations of water and carboxylic end groups in the sample. The equation can also be written as: ln [Mn(t)/Mn(0)]=−Kt. Therefore, by representing data for Mn(t)/Mn(0) versus t on a log-linear plot, one may infer the hydrolytic degradation rate from the slope of the connecting points.

Embodiments of the present invention encompass methods of adjusting the time-dependent degradation behavior of a bioabsorbable polymeric device, such as a stent, and the devices formed. In particular, embodiments encompass bioabsorbable polymeric devices of a high molecular weight polymer. In some embodiments, the bioabsorbable polymeric device is formed from a solvent based process. As used herein, a “high molecular weight polymer,” when used in referring to the polymer of a polymeric stent scaffolding or polymeric device body (and particularly a bioabsorbable polymeric stent scaffolding or bioabsorbable polymer device body) refers to polymer to which at least one of the following conditions applies: (a) the polymer has an inherent viscosity of at least 3.3 dl/g in chloroform at 25° C.; (b) the polymer has a number average molecular weight greater than 250,000 g/mole; (c) the polymer has a weight average molecular weight greater than 280,000 g/mole. In some embodiments, the bioabsorbable polymer has an inherent viscosity of at least 4.0 dl/g, at least 4.5 dl/g, at least 5.0 dl/g, at least 6.0 dl/g, or at least 7.0 dl/g in chloroform at 25° C. In some embodiments, the inherent viscosity is at least 8.0 dl/g in chloroform. For the polymer, the upper limit of inherent viscosity may be 25 dl/g, 15 dl/g, or 10 dl/g in chloroform at 25° C. In some embodiments, the polymer may have a number average molecular weight not greater than 1,200,000 g/mole, the polymer may have a weight average molecular weight of not greater than 1,500,000 g/mole, or both. In some embodiments, the polymer has a number average molecular weight greater than 275,000 g/mole, greater than 300,000 g/mole, greater than 350,000 g/mole, greater than 400,000 g/mole, greater than 500,000 g/mole, greater than 600,000 g/mole, or greater than 750,000 g/mole, but not greater than 2,500,000 g/mole. In some embodiments, the polymer has a weight average molecular weight greater than 300,000 g/mole, greater than 350,000 g/mole, greater than 400,000 g/mole, greater than 450,000 g/mole, greater than 500,000 g/mole, greater than 675,000 g/mole, or greater than 800,000 g/mole, but not greater than 3,000,000 g/mole. In some embodiments, number average molecular weight (Mn) and weight average molecular weight (Mw) may be determined by Gel Permeation Chromatography (GPC) using polystyrene standards.

In some embodiments, the polymer of the scaffold has a crystallinity between 0.2% and 65%. In some embodiments, the polymer has a crystallinity between 0.2% and 50%. In some embodiments, the polymer has a crystallinity between 0.2% and 45%. In some embodiments, the polymer has a crystallinity between 0.2% and 40%. In some embodiments, the polymer has a crystallinity between 0.1% and 35%. In some embodiments, the polymer has a crystallinity between 0.1% and 30%. In some embodiments, the polymer has a crystallinity between 0.1% and 25%. In some embodiments, the polymer has a crystallinity between 0.1% and 20%.

In some embodiments, the polymer used in forming the device body includes an additive to increase the rate of degradation of the polymer. In some embodiments, the additive is the, or at least one, constituent monomer of the bioabsorbable polymer of the device body. Conventionally, polymerization is performed to result in a product with a monomer content as low as possible. Additionally, monomer extraction conventionally is applied to remove all monomer or as much as practically possible from a polymer. The additive which is the, or at least one, constituent monomer of the bioabsorbable polymer of the device body may be present at a level of 0.001 to 0.06 weight fraction, where the weight fraction is the weight of the additive to the sum of the weight of the bioabsorbable polymer and weight of all additives that are the, or at least one, constituent monomer of the bioabsorbable polymer of the device body, and the sum excludes other materials such as additional polymers, drugs, particles, etc. In some embodiments, the additive is the, or at least one, constituent monomer of the bioabsorbable polymer of the device body, and the additive is present at a weight fraction of 0.002 to 0.05, 0.005 to 0.05, 0.01 to 0.05, 0.02 to 0.05, or 0.025 to 0.05 weight fraction as defined above. In some embodiments, the additive is selected from D,L-lactide (meso-lactide), D,D-lactide, L,L-lactide, glycolide, caprolactone, trimethylene carbonate, p-dioxanone, γ-valeroactone, γ-undecalactone, β-methyl-δ-valerolactone, anhydrides, orthocarbonates, phosphazenes, orthoesters, and amino acids.

It has been observed (see United States Patent Application Publication No. 2011-0021717 A1, published on Jan. 27, 2011) from in vitro and in vivo degradation studies of poly(L-lactide) stents with L-lactide monomer that L-lactide, when used as an additive to a device body of poly(L-lactide), provides a dramatic and unexpected increase in the degradation rate of the stent, particularly above about a weight fraction of 0.005 (weight fraction is weight of additive divided by the sum of the weight of the additive and the weight of the bioabsorbable polymer). Stents having monomer compositions above about 0.005 weight fraction of L-lactide blended with poly(L-lactide) lose mechanical strength, lose mechanical integrity, and erode away in a fast way. Additionally, the low concentration of the lactide monomer are advantageous since the effect of the dispersed monomer in the polymer has no or a minimal effect on the mechanical properties of the poly(L-lactide) polymer.

In some embodiments, the additive is an oligomer of the, or at least one, constituent monomer of the bioabsorbable polymer. As a non-limiting example, for poly(L-lactide), low molecular weight oligomers of poly(L-lactide) can also increase the degradation rate, and thus adjust degradation behavior. However, the increase is primarily due to acidic end groups that act as catalysts to increase degradation rate of the poly(L-lactide). Thus, the larger the oligomer, a higher weight fraction of oligomer in the poly(L-lactide) is required to have the same effect on the degradation rate. Therefore, a much lower weight fraction of L-lactide monomer than given oligomer is required for a similar effect as the oligomer. If the weight fraction of the oligomer is too high, it may negatively impact the mechanical properties of the stent. Thus, in some embodiments, oligomers with an number average molecular weight of about equal to 1,000 g/mol, equal to 1,000 g/mol, less than 1,000 g/mol, or a combination thereof, may be used as the additive, and the weight fraction of the oligomer, where the weight fraction is the weight of the additive divided by the sum of the weight of the bioabsorbable polymer and weight of all additives that are oligomers of the or at least one constituent monomer of the bioabsorbable polymer of the device body, and the sum excludes other materials such as additional polymers, drugs, particles, etc., may be about 0.02 to about 0.25, or 0.04 to 0.25. In some embodiments, oligomers with an number average molecular weight of about equal to 1,000 g/mol, equal to 1,000 g/mol, less than 1,000 g/mol, or a combination thereof may be used as the additive, and the weight fraction of the oligomer may be about 0.03 to about 0.25, or 0.03 to 0.25, or about 0.04 to about 0.20, or 0.04 to 0.20. In some embodiments, the oligomer is not smaller than a trimer, or not smaller than four constitutional units. In some embodiments, the oligomer includes dimers and trimers as well as oligomers of a greater number of constitutional units.

In some embodiments, the additive may be a free hydroxy acid, such as, without limitation, L-lactic acid or glycolic acid, or an oligomer thereof. Non-limiting examples of hydroxy acids which may be used as an additive include L-lactic acid, D-lactic acid, glycolic acid, 2-hydroxybutyric acid, 3-hydroxybutyric acid, 2-hydroxyvaleric acid, 3-hydroxybutyric acid, 4-hydroxybutyric acid, 3-hydroxyvaleric acid, 4-hydroxyvaleric acid, 5-hydroxyvaleric acid, dimethylglycolic acid, β-hydroxypropanic acid, α-hydroxybutyric acid, α-hydroxycaproic acid, β-hydroxycaproic acid, γ-hydroxycaproic acid, δ-hydroxycaproic acid, δ-hydroxymethylcaproic acid, ε-hydroxycaproic acid, ε-hydroxymethylcaproic acid, citric acid, and tartaric acid. Additional examples include all hydrocarbon hydroxyl-carboxylic acids of 3 to 16 carbon atoms including linear, branched, cyclic, and aromatic compounds. In some embodiments, the oligomer a free hydroxy acid is not smaller than a trimer, or not smaller than 4 constitutional units, and not more than 50 constitutional units. In some embodiments, the oligomer includes dimers and trimers as well as oligomers of a greater number of constitutional units.

In some embodiments, the additive may be an ester of a hydroxy acid, including cyclic esters and di-esters. Examples include, without limitation, D,L-lactide (meso-lactide), D,D-lactide, L,L-lactide, glycolide, caprolactone, trimethylene carbonate, p-dioxanone, γ-valeroactone, γ-undecalactone, β-methyl-δ-valerolactone, and combinations thereof. Additional examples include all hydrocarbon esters of 1 to 16 carbon atoms, whether linear, branched, cyclic or aromatic, of all of the above mentioned hydroxy acids (D-lactic acid, L-lactic acid, glycolic acid, etc.) and oligomers thereof. In some embodiments, if the additive is an ester of an oligomer of a hydroxy acid, the oligomer is not more than 20 constitutional units, or not more than 10 constitutional units. Other non-limiting examples include the methyl, ethyl, n-propyl, isopropyl, butyl, sec-butyl, iso-butyl, pentyl, and hexyl esters of both L-lactic, D-lactic acid, and their oligomers.

In some embodiments, an ester of a hydroxy acid, a di-ester of a hydroxy acid, the hydroxy acid itself, or a combination thereof, is the or at least one constituent monomer of the bioabsorbable polymer, and the additive is a hydroxy acid, ester of a hydroxy acid, di-ester of hydroxy acid, oligomers of any of the preceding, or a combination thereof, where the hydroxy acid is different from any hydroxy acid that is a constituent monomer of the bioabsorbable polymer, or the hydroxy acid of an ester of a hydroxy acid or a di-ester of a hydroxy acid that is a constituent monomer of the bioabsorbable polymer. As a non-limiting example, if the bioabsorbable polymer is poly(L-lactide), then the additive may be glycolic acid.

In some embodiments, the additive is a fatty acid, an ester of a fatty acid, or a combination thereof. Non-limiting examples of fatty acids include acetic acid, propanoic acid, butyric acid, caprylic acid, caproic acid, capric acid, lauric acid, myristic acid, palmitic acid, stearic acid, arachidic acid, behenic acid, lignoceric acid, and cerotic acid. Non-limiting examples of esters of fatty acids include all hydrocarbon esters of 1 to 16 carbon atoms, whether linear, branched, cyclic or aromatic, of all of the above mentioned fatty acids. If the additive is a fatty acid, an ester of a fatty acid, or a combination thereof, the weight fraction may be from about 0.002 to about 0.03, preferably from about 0.005 to about 0.03, and more preferably, from 0.01 to 0.03, where the weight fraction of the additive is the weight of the additive to the sum of the bioabsorbable polymer and all additives which are fatty acids and esters of fatty acids, and the sum excludes other materials such as additional polymers, drugs, particles, etc.

In some embodiments, the additive is an unsaturated fatty acid, an ester of an unsaturated fatty ester, or a combination thereof. Non-limiting examples of unsaturated fatty acids include myristoleic acid, palmitoleic acid, sapienic acid, oleic acid, elaidic acid, vaccenic acid, linoleic acid, linoelaidic acid, alpha-linolenic acid, arachidonic acid, eicosapentaenoic acid, erucic acid, and docosahexaenoic acid. Non-limiting examples of esters of unsaturated fatty acids include all hydrocarbon esters of 1 to 16 carbon atoms, whether linear, branched, cyclic or aromatic, of all of the above mentioned unsaturated fatty acids.

In some embodiments, the additive is a dicarboxylic acid, an ester of a dicarboxylic acid, or a combination thereof. Non-limiting examples of dicarboxylic acid include oxalic acid, malonic acid, succinic acid, glutaric acid, adipic acid, pimelic acid, suberic acid, azelaic acid, sebacic acid, ortho-phthalic acid, isophthalic acid, and terephthalic acid. Additional examples include all dicarboxylic hydrocarbon acids with 2 to 12 carbon atoms. Non-limiting examples of esters of dicarboxylic acids include all hydrocarbon esters of 1 to 16 carbon atoms, whether linear, branched, cyclic or aromatic, of all of the above mentioned dicarboxylic acids.

In some embodiments, the additive is an anhydride. Non-limiting examples of anhydrides that may be used as additives include succinic anhydride, glutaric anhydride, maleic anhydride, acetic anhydride, propanoic anhydride, butyric anhydride, valeric anhydride, caproic anhydride, heptanoic anhydride, phthalic anhydride, and benzoic anhydride.

In some embodiments, the additive is an acid selected from the group consisting of citric acid, ascorbic acid, erythorbic acid, thiodipropionic acid, cholic acid, desoxycholic acid, glycocholic acid, taurocholic acid, aspartic acid, tartaric acid, and glutamic acid. Other non-limiting examples of additives include all hydrocarbon esters of 1 to 16 carbon atoms, whether linear, branched, cyclic or aromatic, of all of the above mentioned acids.

In some embodiments, the additive is a metal ion, such as magnesium, calcium, sodium, aluminum, zinc, aluminum, tin, and iron, and salts thereof. If the additive is a metal ion, a salt thereof, or a combination thereof, the weight fraction of the additive, that is weight of the additive to the sum of the weight of the bioabsorbable polymer and all additives which are metal ions, and the sum excludes other materials such as additional polymers, drugs, particles, etc., may be 0.0002 to 0.03, preferably, 0.0005 to 0.025, and more preferably, 0.001 to 0.02.

In some embodiments, the additive is a hygroscopic substance. In general, a “hygroscopic substance” is a substance which absorbs water from its surroundings. As used herein, a “hygroscopic substance” is one which absorbs water such that the substance comprises at least 4.0 weight % water after 60 minutes in an environment of 50% humidity at a temperature of 22° C.±2° C. Examples include, without limitation, substances such as polypropylene glycol and glycerol, and polymers and oligomers such as poly(ethylene glycol), poly(ethylene oxide), polyvinylpyrrolidone (PVP), cellulose, cellulose sulfate, hydroxyl cellulose, hydroxyethylcellulose, gelatin, starch, modified starches, such as hydroxyethyl starch and 2-O-acetyl ethyl cellulose, cellulose acetate, carboxymethyl cellulose (CMC), sodium carboxymethyl cellulose, hydroxypropyl cellulose, hydroxypropyl methylcellulose, methyl cellulose, hydroxyethyl methyl cellulose, and poly[N-(2-hydroxypropyl)-methacrylamide] (poly(HPMA))). For the polymers and oligomers cited above as hygroscopic substances, in some embodiments, the number average molecular weight is greater than 150 g/mole, but not greater than 10,000 g/mole, not greater than 5,000 g/mole, or not greater than 1200 g/mole. Other polymers include block copolymers, including di-block copolymers and tri-block copolymers, of polyethylene glycol, where polyethylene glycol is at least 30 weight % of the block copolymer (and not more than 90 weight %), and bioabsorbable polymers such as, but not limited to, those recited above, and specifically including polylactide, polyglycolide, polycaprolactone, and poly(lactide-co-glycolide) where the lactide may be D-lactide, L-lactide, meso-lactide, D,L-lactide, or a combination thereof. Other polymers include block copolymers of polyethylene oxide and polypropylene oxide, most of which are surfactants. The term “poloxamer” (CAS no. 9003-11-6) refers to tri-block copolymers with a central block of polypropylene oxide) (PPO) and with a block of poly(ethylene oxide) (PEO) on each side where the PEO blocks are usually of the same length in terms of number of constitutional units. These polymers have the formula: HO(C₂H₄0)ₐ(C₃H₆0)_{b}(C₂H₄0)ₐH where “a” and “b” denote the number of polyethylene oxide and polypropylene oxide units, respectively. Poloxamers of types 124, 188, 237, 338, and 407 are specified by a monograph in the National Formulary. Preferred hydrophilic polymers include poloxamers 108, 188, 217, 238, 288, 338, and 407. Some PLURONIC® polymers sold by BASF also meet one of the NF specifications for a type of poloxamer.

Other examples of hygroscopic substances that may be used as additives are sodium biphosphate, sodium pyrophosphate, potassium phosphate, sodium carbonate, sodium bicarbonate, potassium carbonate, sodium sulfate, magnesium sulfate, sodium chloride, potassium chloride, magnesium chloride, sodium phosphate, calcium ascorbate, calcium propionate, calcium sorbate, calcium carbonate, calcium citrate, calcium glycerophosphate, calcium oxide, calcium pantothenate, calcium phosphate, calcium pyrophosphate, calcium sulfate, calcium chloride, calcium gluconate, calcium hydroxide, calcium lactate, and calcium oxide. These salts may be added as small particles. The size of such particles can be less than 100 nm, between 100 nm and 200 nm, or greater than 200 nm, where size can refer to diameter or some other characteristic length. In some embodiments, the size is the diameter as determined by photon correlation spectroscopy (PCS) (often used to determine particle size distributions, and it determines a “Z average” diameter which is close to the volume average diameter). In some embodiments, the size of the particles is selected to be not more than 1/15^{th} of the thickness of a strut or the wall thickness of the device, such as, without limitation, not more than 1/15^{th} of 50 to 300 microns. In some embodiments, the size of the particles is selected to be not more than 1/20^{th} or 1/25^{th} of the thickness of a strut or the wall thickness of the device, such as, without limitation, not more than 1/20^{th} or 1/25^{th} of 50 to 300 microns.

In some embodiments, the additive is small particles (10 nm to 1000 nm) of NaO₂, KO₂, superoxide salts, or a combination thereof. These compounds are insoluble in organics but will cleave ester bonds quite actively when hydrated so as to decrease molecular weight (both weight average and number average) of the bioabsorbable polymer.

In other embodiments, the bioabsorbable polymer has a high polydispersity index. The high polydispersity ensures that some lower molecular weight species are present. In some embodiments, the polydispersity index is 3.2 or greater than 3.2, or preferably 4 or greater than 4. In some embodiments, the polydispersity index is preferably 5 or greater than 5, or 6 or greater than 6. In some embodiments, including those above, the polydispersity index is not more than 10, or not more than 20. The polydispersity index for a polymer is the ratio of the weight average molecular weight to the number average molecular weight.

In other embodiments, the bioabsorbable polymer is blended with a polymer with a higher degradation rate. In some embodiments, the higher degradation polymer is at least 1 weight % of the total of the bioabsorbable polymer and the higher degradation polymer, and up to 8 weight % (wt %) of the total of the bioabsorbable polymer and the higher degradation polymer, preferably, up to 12 wt %, and even more preferably, up to 20%. In some embodiments, the higher degradation polymer has an inherent viscosity, number average molecular weight, weight average molecular weight, or any combination thereof, within at least one of the ranges disclosed above for the bioabsorbable polymer. In some embodiments, the number average molecular weight, the weight average molecular weight, or both, of the higher degradation polymer is less than one-half of that of the bioabsorbable polymer, but more than one-tenth. In other embodiments, the number average molecular weight, the weight average molecular weight, or both, of the higher degradation polymer is less than one fifth, or less than one tenth of that of the bioabsorbable polymer, but more than one hundredth of that of the bioabsorbable polymer. In some embodiments, the bioabsorbable polymer is poly(L-lactide), a copolymer where one constituent monomer is L-lactide, or a combination thereof; and the high degradation polymer, is poly(glycolide), a copolymer where one constituent monomer is glycolide, poly(D,L-lactide), a polymer where the constituent monomers are D-lactide and L-lactide, polydioxanone, poly(4-hydroxybutyrate), poly(trimethylene carbonate), a copolymer where at least one constituent monomer is polydioxanone, poly(4-hydroxybutyrate), poly(trimethylene carbonate), or a combination thereof. As a specific non-limiting example, the bioabsorbable polymer is poly(L-lactide) and the higher degradation polymer blended with the bioabsorbable polymer is poly(D,L-lactide-co-glycolide) of a 50:50 molar ratio of lactide to glycolide.

Embodiments of the present invention encompass use of the above modifications individually, and in combination. As a non-limiting example, the bioabsorbable polymer may have a high polydispersity index, and one or more of the above additives may be used. Embodiments encompass multiple additives from the same class, for example, a combination of hydroxy acids, as well as a combination of one or more additives from one class with one or more additives from one or more other classes, where some substances may belong to more than one class. Embodiments also encompass a device body formed of only bioabsorbable polymer (one or more) and additives (one or more), or a device body with 90 weight % or 95 weight %, and up to 99.99 weight %, being bioabsorbable polymer (one or more) and additives (one or more). Embodiments also encompass a device body consisting essentially of bioabsorbable polymer (one or more) and additive (one or more), where consisting essentially of includes impurities and/or other materials of the bioabsorbable polymer and the additive which are not separately and specifically added to the composition. In some embodiments, with respect to the weight fraction of the additives, the total weight fraction of additives which includes the sum of all the weights of the additives to the sum of weights of all the additives and the weight of the bioabsorbable polymer but excluding the weight of any high degradation polymers as well as drugs, particles, and the like, may be between 0.0005 to 0.05, 0.001 to 0.045, 0.002 to 0.04, 0.0005 to 0.03, 0.0005 to 0.02, 0.0005 to 0.02, and 0.0005 to 0.01. In some embodiments, with respect to the weight fraction of the additives, the total weight fraction of additives which includes the sum of all the weights of the additives to the sum of weights of all the additives and the weight of the bioabsorbable polymer and including the weight of any high degradation polymers, but excluding the weight drugs, particles, and the like, may be between 0.0005 to 0.05, 0.001 to 0.045, 0.002 to 0.04, 0.0005 to 0.03, 0.0005 to 0.02, 0.0005 to 0.02, and 0.0005 to 0.01. In some embodiments, if one or more of the additives is in a class for which a weight fraction is specifically specified herein, with respect to those additives, the specific weight fractions may be applicable, and the above limitations may be applicable to the total of the additives. In some embodiments, if a combination of additives is used, the weight fraction of an individual additive or those of an individual class may be below the more specific weight fractions for that class of additives.

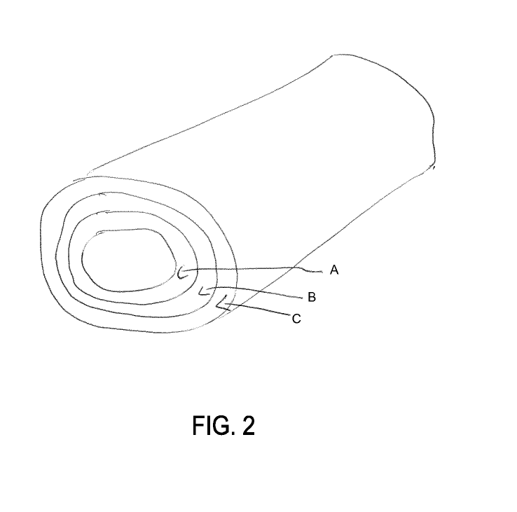

The additives, high degradation polymer, or both may be uniformly distributed in the bioabsorbable polymer or the bioabsorbable polymer and other substances such as drugs, etc. In some embodiments, the additives, high degradation polymer, or both are distributed in a non-homogeneous manner. As an example, a tube which may be patterned to form a stent body or scaffolding with layers A, B, and C, of different material is shown FIG. 2, which is a tube having a wall with concentric layers of different material. The layers may be formed by different methods. Thus, each layer may include a different modification, or no modification, provided that at least part of the stent includes a modification. As a non-limiting example of a device or stent of three layers, the outer and inner layers may be bioabsorbable polymer without additives or other modifications, while the middle layer includes a modification as described above. For example, the middle layer may be formed of a bioabsorbable polymer of a high polydispersity index, may include a higher degradation polymer, may include an additive, or any combination thereof. In some embodiments, such as but not limited to, those described in the above paragraphs, the additive is homogeneously, or substantially homogeneously, distributed within the device body or scaffolding. In some embodiments, the device body is coated, and the coating is initially (as determined within 24 hours of manufacture) free of, or substantially free of (0.2 weight % or less than 0.2 weight % of the coating), the additive. In some embodiments, the device body is coated, and no additive is included in any of the coating materials applied to the device body.

Embodiments of the present invention also encompass medical devices, such as stents, formed of a bioabsorbable polymer with an inherent viscosity of at least 7.0 dl/g, but not more than 25 dl/g, in chloroform at 25° C., with a number average molecular weight greater than 750,000 g/mole, but not greater than 3,000,000 g/mole, as measured by GPC using polystyrene standards, or both, where the time frame for in-vivo mass loss in a human being of at least 90 weight %, and in some embodiments, at least 95 weight % (when compared to the initial mass), is in the range of 16 months to 38 months, preferably, in the range of 20 months to 36 months, and in some embodiments, in the range of 22 months to 30 months. Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, where the time frame for in-vitro mass loss of at least 90 weight %, and in some embodiments, at least 95 weight % (when compared to the initial mass), as determined in phosphate buffered saline at 37° C.±2° C. is in the range of 16 months to 38 months, preferably, in the range of 20 months to 36 months, and in some embodiments, in the range of 22 months to 30 months.

Embodiments of the present invention also encompass medical devices, such as stents, formed of a bioabsorbable polymer with an inherent viscosity of at least 7.7 dl/g, but not more than 25 dl/g, in chloroform at 25° C., with a number average molecular weight greater than 850,000 g/mole, but not greater than 3,000,000 g/mole, as measured by GPC using polystyrene standards, or both, where the time frame for in-vivo mass loss in a human being of at least 90 weight %, and in some embodiments, at least 95 weight % (when compared to the initial mass), is in the range of 16 months to 38 months, preferably, in the range of 20 months to 36 months, and in some embodiments, in the range of 22 months to 30 months. Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole, but not greater than 3,000,000 g/mole, as measured by GPC using polystyrene standards, or both, where the time frame for in-vitro mass loss of at least 90 weight %, and in some embodiments, at least 95 weight % (when compared to the initial mass), as determined in phosphate buffered saline at 37° C.±2° C. is in the range of 16 months to 38 months, preferably, in the range of 20 months to 36 months, and in some embodiments, in the range of 22 months to 30 months.

Embodiments of the present invention also encompass medical devices, such as stents, formed of a bioabsorbable polymer with an inherent viscosity of at least 7.0 dl/g, but not more than 25 dl/g, in chloroform at 25° C., with a number average molecular weight greater than 750,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof, if both are present, is present in a sufficient amount, the bioabsorbable polymer is sufficiently polydisperse, or a combination thereof, that the time frame for in-vivo mass loss in a human being of least 90 weight %, and in some embodiments, at least 95 weight % (when compared to the initial mass), is in the range of 16 months to 38 months, preferably, in the range of 20 months to 36 months, and in some embodiments, in the range of 22 months to 30 months. Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that the time frame for in-vitro mass loss of least 90 weight %, and in some embodiments, at least 95 weight % (when compared to the initial mass), as determined in phosphate buffered saline at 37° C.±2° C. is in the range of 16 months to 38 months, preferably, in the range of 20 months to 36 months, and in some embodiments, in the range of 22 months to 30 months.

Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that the time frame for in-vivo mass loss in a human being of at least of least 90 weight %, and in some embodiments, at least 95 weight % (when compared to the initial mass), is in the range of 16 months to 38 months, preferably, in the range of 20 months to 36 months, and in some embodiments, in the range of 22 months to 30 months. Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that the time frame for in-vitro mass loss of least 90 weight %, and in some embodiments, at least 95 weight % (when compared to the initial mass), as determined in phosphate buffered saline at 37° C.±2° C. is in the range of 16 months to 38 months, preferably, in the range of 20 months to 36 months, and in some embodiments, in the range of 22 months to 30 months.

Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that at 24 months after implantation in-vivo in a human being the number average molecular weight of the bioabsorbable polymer is not more than 40,000 g/mole, and in some embodiments, not more than 20,000 g/mole. Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that at 24 months after being placed in phosphate buffered saline at 37° C.±2° C., the number average molecular weight of the bioabsorbable polymer is not more than 40,000 g/mole, and in some embodiments, not more than 20,000 g/mole.

Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that at 24 months after implantation in-vivo in a human being the number average molecular weight of the bioabsorbable polymer is not more than 40,000 g/mole, and in some embodiments, not more than 20,000 g/mole. Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole, but not greater than 3,000,000 g/mole, as measured by GPC using polystyrene standards, or both, having an additive blended or dispersed in the polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, that at 24 months after being placed in phosphate buffered saline at 37° C.±2° C., the number average molecular weight of the bioabsorbable polymer is not more than 40,000 g/mole, and in some embodiments, not more than 20,000 g/mole.

Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, where at 20 months after implantation in-vivo in a human being the number average molecular weight of the bioabsorbable polymer is not more than 40,000 g/mole, and in some embodiments, not more than 20,000 g/mole. Embodiments of the present invention also encompass medical devices, such as stents, formed of a bioabsorbable polymer with an inherent viscosity of at least 7.0 dl/g, but not greater than 25 dl/g, in chloroform at 25° C., with a number average molecular weight greater than 750,000 g/mole, but not greater than 3,000,000 g/mole, as measured by GPC using polystyrene standards, or both, where at 20 months after being placed in phosphate buffered saline at 37° C.±2° C., the number average molecular weight of the bioabsorbable polymer is not more than 40,000 g/mole, and in some embodiments, not more than 20,000 g/mole.

Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole, but not greater than 3,000,000 g/mole, as measured by GPC using polystyrene standards, or both, where at 20 months after implantation in vivo in a human being the number average molecular weight of the bioabsorbable polymer is not more than 40,000 g/mole, and in some embodiments, not more than 20,000 g/mole. Embodiments of the present invention also encompass medical devices, such as stents, formed of a bioabsorbable polymer with an inherent viscosity of at least 7.7 dl/g, but not greater than 25 dl/g, in chloroform at 25° C., with a number average molecular weight greater than 850,000 g/mole, but not greater than 3,000,000 g/mole, as measured by GPC using polystyrene standards, or both, where at 20 months after being placed in phosphate buffered saline at 37° C.±2° C., the number average molecular weight of the bioabsorbable polymer is not more than 40,000 g/mole, and in some embodiments, not more than 20,000 g/mole.

Embodiments of the present invention also encompass methods of treatment of a patient in need of treatment of a disorder or condition, the treatment comprising implanting within a vascular lumen of the patient a bioabsorbable polymeric device, such as a stent, in which the bioabsorbable polymer of the polymeric device has an inherent viscosity of at least 7.0 dl/g in chloroform at 25° C., has a number average molecular weight greater than 750,000 g/mole, but not greater than 3,000,000 g/mole, as measured by GPC using polystyrene standards, or both, and has an additive blended or dispersed in the bioabsorbable polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, to allow for vasomotion to occur within a time frame ranging from 6 months and 30 months, and preferably, from 9 to 24 months, and in some embodiments, from 12 months to 18 months after implantation.

Embodiments of the present invention also encompass methods of treatment of a patient in need of treatment of a disorder or condition, the treatment comprising implanting within a vascular lumen of the patient a bioabsorbable polymeric device, such as a stent, in which the bioabsorbable polymer of the polymeric device has an inherent viscosity of at least 7.7 dl/g, but not greater than 25 dl/g, in chloroform at 25° C., has a number average molecular weight greater than 850,000 g/mole, but not greater than 3,000,000 g/mole, as measured by GPC using polystyrene standards, or both, and has an additive blended or dispersed in the bioabsorbable polymer, uniformly or non-uniformly, having a second polymer that degrades more quickly than the bioabsorbable polymer blended with the bioabsorbable polymer, uniformly or non-uniformly, having a polydispersity index of 4 or greater than 4, or any combination thereof, such that the additive, second polymer, or combination thereof if both are present, is present in a sufficient amount, the bioabsorbable polymer is sufficiently polydisperse, or a combination thereof, to allow for vasomotion to occur within a time frame ranging from 6 months and 30 months, and preferably, from 9 to 24 months, and in some embodiments, from 12 months to 18 months after implantation. Embodiments of the present invention also encompass methods of treatment of a patient in need of treatment of a disorder or condition, the treatment comprising implanting within a vascular lumen of the patient a bioabsorbable polymeric device, such as a stent, in which the bioabsorbable polymer of the polymeric device has an inherent viscosity of at least 7.0 dl/g in chloroform at 25° C., has a number average molecular weight greater than 750,000 g/mole, but not greater than 3,000,000 g/mole, as measured by GPC using polystyrene standards, or both, and allows for vasomotion to occur within a time frame ranging from 6 months and 30 months, and preferably, from 9 to 24 months, and in some embodiments, from 12 months to 18 months after implantation. Embodiments of the present invention also encompass methods of treatment of a patient in need of treatment of a disorder or condition, the treatment comprising implanting within a vascular lumen of the patient a bioabsorbable polymeric device, such as a stent, in which the bioabsorbable polymer of the polymeric device has an inherent viscosity of at least 7.7 dl/g, but not greater than 25 dl/g, in chloroform at 25° C., has a number average molecular weight greater than 850,000 g/mole, but not greater than 3,000,000 g/mole, as measured by GPC using polystyrene standards, or both, and allows for vasomotion to occur within a time frame ranging from 6 months and 30 months, and preferably, from 9 to 24 months, and in some embodiments, from 12 months to 18 months after implantation.

Embodiments of the present invention also include methods of forming a polymeric device, specifically a polymeric stent. As noted above, some processes such as melt extrusion and radiation sterilization result in a decrease in the molecular weight of the polymer. Thus, in some embodiments, the formation of a polymer construct, such as a tube, from which the device, such as a stent, is formed using solvent processing methods. Solvent processing generally refers to forming a polymer construct such as a tube from solution of polymer dissolved in a solvent. Non-limiting examples of solvent processing methods include spray coating, gel extrusion, supercritical fluid extrusion, roll coating and dip coating. In some embodiments, the polymer construct, such as a tube, is formed by ram extrusion, compression molding, or both, which result in less polymer degradation than traditional melt processing operations. Moreover, the use of solvent processing typically allows for uniform, or substantially uniform, distribution of the additive, high degrading polymer, or both, in the bioabsorbable polymer because the additive, high degrading polymer, or both, may be dissolved, partially dissolve, dispersed, or a combination thereof, in the solvent. The preferred solvent processing techniques for making stents are described in the text below.

Solvent processing methods include the use of gel extrusion, as described in patent application Ser. No. 11/345,073 (United States Patent Application Publication No. 2007-0179219 A1, published on Aug. 2, 2007), which is incorporated by reference herein in its entirety, and spray coating, which is described in United States Patent Application Publication No. 2010-0262224 A1, published on Oct. 14, 2010, which is incorporated by reference herein in its entirety.

A particularly preferred solvent processing method is dip coating. Dip coating is a method of forming a coating layer onto an object which includes immersing the object in a coating material or solution that includes a polymer dissolved in a solvent, withdrawing the object from the solution, and removing solvent from the solution retained on the surface of the object. Upon removal of the solvent, a layer of polymer is formed on the surface of the object. The steps above can be repeated to form multiple layers of polymer over the object to obtain a desired thickness of a coating layer. With respect to the embodiments of the invention involving an additive, the additive may be dissolved, partially dissolved, dispersed or a combination of dissolved and dispersed in the solution of the bioabsorbable polymer.

The object can be a cylindrical member or mandrel over which a tubular coating layer is formed. The mandrel can be made of any material that is not soluble in the solvent of the polymer solution. In some embodiments, the mandrel is made of a metal such as aluminum or stainless steel. In other embodiments, the mandrel is made from a glass with a polished surface. In some other embodiments, the mandrel is made of a soluble material that is insoluble in the solvent used for the coating. In other embodiments, the mandrel is made of a polymer. The coating layer may be formed so that its radial thickness or the thickness of the wall of the tubular layer is the desired thickness of a stent scaffolding. The coating layer may then be removed from the mandrel and machined to form a stent scaffolding.

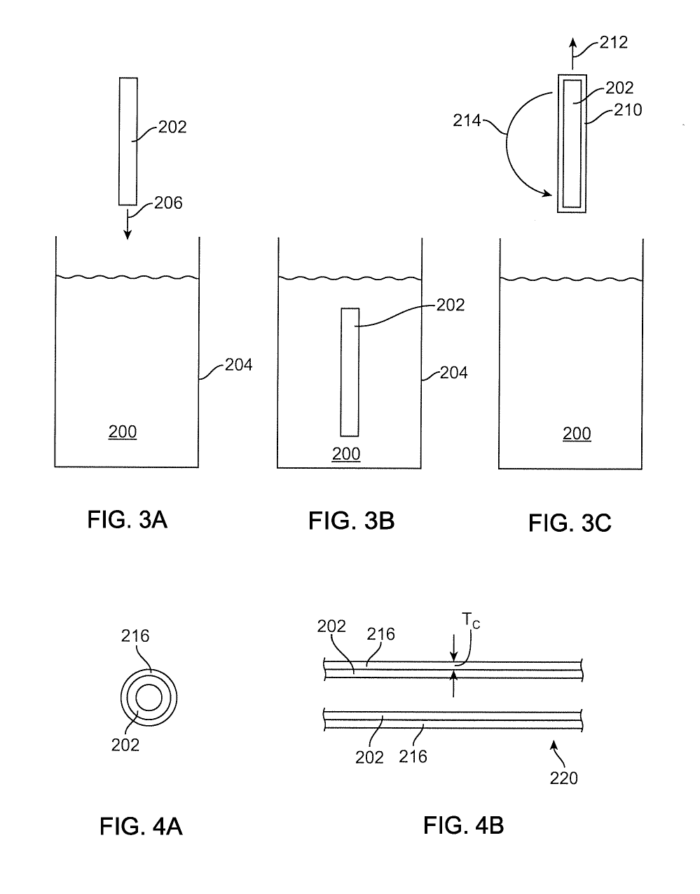

FIGS. 3A-C illustrate the dip coating process of the present invention. As shown in FIG. 3A, a mandrel 202 is lowered, as shown by an arrow 206 into a container 204 having a solution 200 that includes a bioabsorbable polymer dissolved in a solvent, the solution optionally including an additive dissolved, dispersed, or both dissolved and dispersed in the solution of the bioabsorbable polymer. The cylindrical axis of the mandrel is perpendicular to the surface of the solution, although the mandrel can be immersed at an angle different from 90° to the solution surface. As shown in FIG. 3B, at least part of the mandrel remains immersed in solvent 200 for a selected time or dwell time. Referring to FIG. 3C, mandrel 202 is then removed from solvent 200 as shown by an arrow 212. The cylindrical axis of mandrel 202 is perpendicular to the surface of the solution, although the mandrel can be removed at angle different from 90° to the solution surface. The use of a 90° angle is expected to facilitate uniformity in the coating thickness. Solution 210 is retained on mandrel 202 after removal from the solution 200 in container 204. Solvent is then removed from the retained solution 210 which results in the formation of a coating layer of the bioabsorbable polymer, and optionally any additives or other materials. The solvent can be removed using various types of drying methods described below.

Other dip coating processes can be envisioned by those skilled in the art. These include immersing only a small part of the mandrel into the solution and while rotating parallel to the solution. This process helps ensure an even coating thickness.

In another embodiment, a hollow mandrel is dipped into the solution of the bioabsorbable polymer, optionally including an additive, and a vacuum is drawn at one end of the mandrel causing the solution to be drawn into the mandrel. When the mandrel is lifted from the solution, the solution will drain from the inside leaving the inside to the mandrel coated with the bioabsorbable polymer and optionally, the additive.

If the coating layer is a desired thickness, the coating layer can be removed after solvent removal and machined to form stent. Alternatively, the steps in FIGS. 3A-C can be repeated one or more times until a desired thickness of polymer is achieved. In some embodiments, the coated tube can be rotated 180° before each coating step is repeated because gravity causes a greater volume of retained solution near a lower end of the mandrel after removal of the mandrel from the solution. FIGS. 4A and 4B depict radial and axial cross-sections, respectively, of a hollow coated mandrel. FIGS. 4A and 4B show mandrel 202 with a polymer coating layer 216 with a thickness Tc.

There are several parameters in the dip coating process that can affect the quality and uniformity of the coating layer. It is desirable for the tubular coating layer to be uniform circumferentially and along the cylindrical axis. Parameters include the concentration and viscosity of the polymer solution, the dwell time in solution, and the rate of removal of the mandrel from solution.

In some embodiments, polymer concentration can be at or near (within 10%) a saturation concentration. Such concentration is expected to result in the highest viscosity and the thickest coating layer per immersion. Alternatively, polymer concentration can be less than saturation, for example, less than 50% or less than 25% saturation. A more dilute and less viscous solution may result in a coating layer. However, a more dilute solution will require a higher number of repeated coating steps to provide a final desired coating thickness.

The rate of removal of the mandrel from the solution can influence the uniformity of a coating layer of a single coating step and the consistency of thickness of coating layers deposited in separate steps. For the removal time ranges considered in United States Patent Application Publication No. 2010-0262224 A1, the rate of removal is directly proportional to the uniformity of coating layer thickness along the cylindrical axis for a coating from a single step. As the rate decreases, there is a greater difference in coating thickness between the top end and bottom end of the mandrel. In addition, the removal rate is directly proportional to the consistency in thickness between coating layers deposited in separate steps.

The solvent can be removed from the solution retained on the mandrel by methods known in the art including air drying, baking in an oven, or both. In air drying a gas stream is directed on or blown onto the mandrel. The gas can be at room temperature (about 20° C. to about 22° C.) or heated (a temperature in the range of about 30° C. to about 90° C.) to increase the removal rate.

There are various ways to remove the tubular coating layer from the mandrel to further process the coating layer in the fabrication of a stent.

The coating layer can be formed over a mandrel made of a dissolvable material to be used as a dipping mandrel. After forming the coating layer, the mandrel can be dissolved by a solvent for the mandrel material, but that is a non-solvent for the coating polymer. In an exemplary embodiment, the mandrel is a wax and the coating polymer is PLLA.

In another method of removal, the tubular coating layer is formed over a hollow mandrel or pipe with one end of the pipe covered by the coating layer. The polymer is formed such that it wraps around the ends of the pipe, creating a seal. After completing the coating layer, the coating layer can be cut off one end of the polymer wrapped pipe. Compressed air blow into the open end forces the tube off the mandrel.

In another method, the coating layer is laser machined to form the stent pattern while still mounted on the mandrel.

Another method includes forming the coating layer over an inflated tubular balloon. The inflated tubular balloon is dip-coated as described above. A small scale balloon is created that is 3.2 mm inflated outer diameter (OD), and the balloon is dip-coated directly. After dip-coating, the balloon is deflated and removed from the coating layer.

In another removal method, the mandrel is heated after forming the coating layer. The heating is expected to loosen the coating layer, allowing it to be slipped off.

Another removal method includes application of oily or greasy coating over the mandrel before dip coating. Once dip coating is completed, the coating layer is slipped off.

In another method, a flexible rubber sleeve is wrapped around the mandrel prior to dip coating. After coating is complete, the tube may be pulled off the mandrel by the sleeve. The tube is then removed from the sleeve.

In another method, after dip coating over a metal mandrel, the metal mandrel is cooled sufficiently to cause shrinkage of the mandrel, allowing the coating layer to be pulled off.

In another method, after dip coating over a metal mandrel, the metal mandrel is dipped into a solvent or solvent blend which does not dissolve but only swells the coating. This allows the coating layer to be pulled off.

In another method, a mechanical slider may be used to force the tube off of the mandrel.

An automated dip coating system can include a syringe pump that performs a controlled immersion into a polymer solution, dwell time in the polymer solution, and removal from the polymer solution of one or more mandrels. A syringe pump is a device designed to advance the plunger of a syringe at a consistent, precise rate for continuously controlled liquid delivery. A specially adapted mounting system for mandrels can be coupled to the plunger. The motion of the plunger is designed to provide controlled motion that immerses the mandrels at a controlled rate, to allow the mandrels to dwell in the solution for a selected time, and to provide controlled motion that removes the mandrels from the solution at a controlled rate. An exemplary syringe pump for automated dip coating is a Harvard Apparatus PHD 2000 programmable syringe pump.

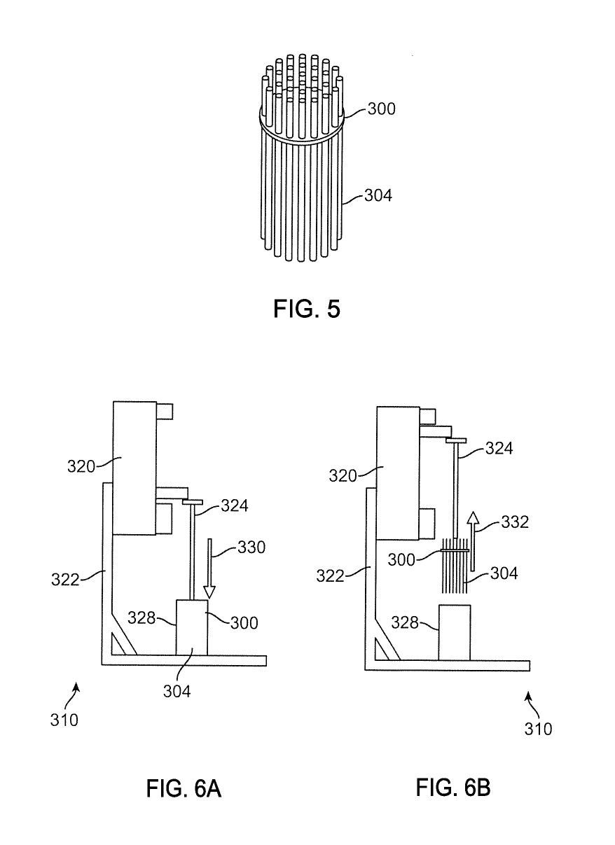

FIG. 5 depicts a mandrel mounting disk 300 having a plurality of holes configured to hold mandrels for a dip coating operation. A plurality of mandrels 304 are mounted on mounting disk 300 within the holes. FIG. 6A depicts a system 310 for controlled dip coating of mandrels 304 mounted on mounting disk 300. System 310 has a syringe pump 320 positioned vertically and supported by a bracket 322. Syringe pump 320 includes a syringe plunger 324 that is coupled to mounting disk 300 on which are mounted a plurality of mandrels 304 (as illustrated in FIG. 5). In FIG. 6A, mounting disk 300 is positioned such that mandrels are immersed in a solvent within a container 328. Plunger 324 is configured to move downward, as shown by an arrow 330, at a controlled rate to immerse the mandrels in the solvent and then allow the mandrels to dwell in the solvent for a selected amount of time. FIG. 6B shows mounting disk 300 and mounted mandrels 304 removed from the solution. Plunger 324 is configured to move upward, as shown by an arrow 332, at a controlled rate to remove the mandrels from the solvent and is further configured to allow the mandrels to remain removed for a period of time to allow for removal of solvent from the solution retained on the mandrels.

One advantage of using the high molecular weight material for a stent body is that a stent body machined from the tube as-formed may have sufficient mechanical properties to support a bodily lumen without further processing. Further processing includes, but is not limited to, a radial expansion step to improve properties such as radial strength, modulus, and fracture toughness. In some embodiments, there is no radial expansion operation, while in other embodiments, the polymer tube is radially expanded. The radial expansion can be accomplished by a blow molding process. In such a process, the polymer tube is disposed within a cylindrical mold with a diameter greater than the polymer tube. The polymer tube is heated, preferably so that its temperature is above its glass transition temperature (Tg). The pressure inside of the tube is increased to cause radial expansion of the tube so the outside surface of the tube conforms to the inside surface of the mold. The polymer tube is then cooled below Tg and further processing steps can then be performed.

A polymer tube, whether radially expanded or not, may be subject to an operation, such as laser machining or chemical etching, to form a pattern in the tube thus forming the stent.

In preferred embodiments, the device body, preferably a stent, is of the bioabsorbable polymer poly(L-lactide) (PLLA), a polymer with L-lactide, L-lactic acid, or both, as a constituent monomer of at least 30 mol %, preferably, at least 50 mol %, more preferably 60 mol %, and even more preferably at least 70 mol %, and up to 98 mol %, or a combination thereof, where the bioabsorbable polymer has an inherent viscosity of at least 3.3 dl/g, a number average molecular weight greater than 250,000 g/mole as measured by GPC using polystyrene standards, or both, and a crystallinity of 45% or less, preferably 40% or less, more preferably 35% or less, even more preferably 30% or less, and most preferably, 25% or less, but at least 0.1% crystallinity, and an additive, a higher degradation polymer, or both. Thus, in some embodiments, the device body is formed from poly(L-lactide), and the additive is the constituent monomer, L-lactide. In some embodiments, the device body poly(L-lactide-co-glycolide), and the additive is L-lactide, glycolide, or a combination thereof, that is at least one of the constituent monomers or a combination thereof. In preferred embodiments, the device body is formed using solvent processing methods.

In some embodiments, the device body, preferably a stent, is of the bioabsorbable polymer poly(L-lactide) (PLLA), a polymer with L-lactide, L-lactic acid, or both, as a constituent monomer, or a combination thereof, and the additives L-lactide, D-lactide, D,L-lactide, meso-lactide, glycolide, L-lactic acid, D-lactic acid, glycolic acid, and their oligomers are expressly excluded as being additives. In some embodiments, the bioabsorbable polymer is poly(L-lactide) (PLLA), and L-lactide is expressly excluded as an additive.

The stent can further include a coating of one or multiple layers disposed over the body or scaffolding having dimension of about 30 angstroms to 20 microns, preferably 30 angstroms to 10 microns, and more preferably 150 angstroms to 5 microns. In one embodiment, the coating can be a polymer and drug mixture. For example, the coating can be poly(D,L-lactide) and the drug could be an antiproliferative such as everolimus.

The coating can be free of the additives other than incidental migration or diffusion of the additives into the coating. Everolimus may be included in the device body.

Other drugs that may be used in a coating over the device body, within the device body, or both. Drugs that may be suitable for use in the embodiments of the present invention, individually or in combination, depending, of course, on the specific disease being treated, include, without limitation, anti-restenosis, pro- or anti-proliferative, anti-inflammatory, anti-neoplastic, antimitotic, anti-platelet, anticoagulant, antifibrin, antithrombin, cytostatic, antibiotic, anti-enzymatic, anti-metabolic, angiogenic, cytoprotective, angiotensin converting enzyme (ACE) inhibiting, angiotensin II receptor antagonizing, and cardioprotective drugs. Some drugs fall into more than one category.

The term “anti-proliferative” as used herein, refers to a therapeutic agent that works to block the proliferative phase of acute cellular rejection. The anti-proliferative drug can be a natural proteineous substance such as a cytotoxin or a synthetic molecule. Other drugs include, without limitation, anti-proliferative substances such as actinomycin D, and derivatives thereof (manufactured by Sigma-Aldrich 1001 West Saint Paul Avenue, Milwaukee, Wis. 53233; or COSMEGEN™ available from Merck) (synonyms of actinomycin D include dactinomycin, actinomycin IV, actinomycin I1, actinomycin X1, and actinomycin C1), all taxoids such as taxols, docetaxel, paclitaxel, and paclitaxel derivatives, FKBP-12 mediated mTOR inhibitors, and pirfenidone. Other anti-proliferative drugs include rapamycin (sirolimus), everolimus, zotarolimus (ABT-578), biolimus A9, ridaforolimus (formerly deforolimus, and also known as AP23573), tacrolimus, temsirolimus, pimecrolimus, novolimus, myolimus, umirolimus, merilimus, 40-O-(3-hydroxypropyl)rapamycin, 40-O-[2-(2-hydroxyl)ethoxy]ethyl-rapamycin, 40-O-tetrazolylrapamycin, and 40-epi-(N1-tetrazolyl)-rapamycin. Other compounds that may be used as drugs are compounds having the structure of rapamycin but with a substituent at the carbon corresponding to the 42 or 40 carbon (see structure below).

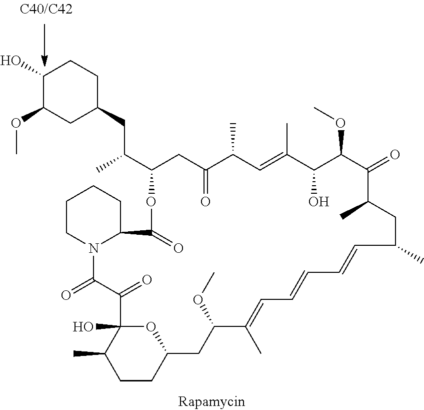 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Additional examples of cytostatic or antiproliferative drugs include, without limitation, angiopeptin, and fibroblast growth factor (FGF) antagonists.

Examples of anti-inflammatory drugs include both steroidal and non-steroidal (NSAID) anti-inflammatories such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexamethasone phosphate, mometasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus and pimecrolimus.

Alternatively, the anti-inflammatory drug can be a biological inhibitor of pro-inflammatory signaling molecules. Anti-inflammatory drugs may be bioactive substances including antibodies to such biological inflammatory signaling molecules.

Examples of antineoplastics and antimitotics include, without limitation, paclitaxel, docetaxel, methotrexate, azathioprine, vincristine, vinblastine, fluorouracil, doxorubicin hydrochloride and mitomycin.

Examples of anti-platelet, anticoagulant, antifibrin, and antithrombin drugs include, without limitation, heparin, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as ANGIOMAX® (bivalirudin), calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide, nitric oxide donors, super oxide dismutases, super oxide dismutase mimetic and 4-amino-2,2,6,6-tetramethylpiperidine-1-oxyl(4-amino-TEMPO).

Examples of ACE inhibitors include, without limitation, quinapril, perindopril, ramipril, captopril, benazepril, trandolapril, fosinopril, lisinopril, moexipril and enalapril.

Examples of angiotensin II receptor antagonists include, without limitation, irbesartan and losartan.

Other drugs that may be used, include, without limitation, estradiol, 17-beta-estradiol, γ-hiridun, imatinib mesylate, midostaurin, feno fibrate, and feno fibric acid.

Other drugs that have not been specifically listed may also be used. Some drugs may fall into more than one of the above mentioned categories. Prodrugs thereof, co-drugs thereof, and combinations thereof of the above listed drugs are also encompassed in the various embodiments of the present invention.

Representative examples of polymers, oligomers, and materials that may be used, individually or in combination, in the a coatings described herein, and optionally, if bioabsorbable, may be used, individually or in combination with any other bioabsorbable material described herein, in forming a device body, include, without limitation, polyesters, polyhydroxyalkanoates, poly(3-hydroxyvalerate), poly(D,L-lactide-co-glycolide), poly(L-lactide-co-glycolide), poly(3-hydroxybutyrate), poly(4-hydroxybutyrate), poly(3-hydroxybutyrate-co-3-hydroxyvalerate), polyhydroxybutyrate, polyhydroxybutyrate-co-hydroxyvalerates, polyhydroxybutyrate-co-hydroxyhexanoate, polyorthoesters, polyanhydrides, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(D-lactide-co-caprolactone), poly(D-lactide), poly(glycolide-co-caprolactone), poly(trimethylene carbonate), polyester amides, poly(glycolic acid-co-trimethylene carbonate), poly(amino acid)s, polyphosphazenes, polycarbonates, cellulose acetate, cellulose butyrate, cellulose acetate butyrate, cellophane, cellulose nitrate, cellulose propionate, cellulose ethers, silk-elastin, elastin mimetic peptides, alginic acid, alginate, chondroitin sulfate, chitosan, chitosan sulfate, collagen, fibrin, fibrinogen, cellulose, cellulose sulfate, carboxymethylcellulose, hydroxyl cellulose, hydroxypropyl cellulose, hydroxypropyl methylcellulose (HPMC), carboxymethylcellulose sodium, hydroxyethylcellulose, gelatin, sugars, starch, modified starches, such as hydroxyethyl starch and 2-O-acetyl starches), polysaccharides, dextran sulfate, dextran, dextrin, xanthan, hyaluronic acid, fragments of hyaluronic acid, polysaccharides, and copolymers thereof.

As used herein, the terms poly(D,L-lactide), poly(L-lactide), poly(D,L-lactide-co-glycolide), and poly(L-lactide-co-glycolide) are used interchangeably with the terms poly(D,L-lactic acid), poly(L-lactic acid), poly(D,L-lactic acid-co-glycolic acid), and poly(L-lactic acid-co-glycolic acid), respectively.

As used herein, caprolactone includes, but is not limited to, ε-caprolactone.

For the purposes of the present invention, the following terms and definitions apply:

“Molecular weight” can refer to the molecular weight of individual segments, blocks, or polymer chains. “Molecular weight” can also refer to weight average molecular weight or number average molecular weight of types of segments, blocks, or polymer chains.

The number average molecular weight (Mn) is the common, mean, average of the molecular weights of the individual segments, blocks, or polymer chains. It is determined by measuring the molecular weight of N polymer molecules, summing the weights, and dividing by N:

$$Mn = \frac{{\sum }_{i} NiMi}{{\sum }_{i} Ni}$$

where Ni is the number of polymer molecules with molecular weight Mi. The weight average molecular weight is given by:

$$Mw = \frac{{\sum }_{i} {NiMi}^{2}}{{\sum }_{i} NiMi}$$

where Ni is the number of molecules of molecular weight Mi.

The “polydispersity” or “polydispersity index” is the ratio Mw/Mn.

The “inherent viscosity” (of a polymer) is the ratio of the natural logarithm of the relative viscosity, ηr, to the mass concentration of the polymer, c, i.e. ηinh=(ln ηr)/c, where the relative viscosity (ηr) is the ratio of the viscosity of a polymer solution, η, to the viscosity of the solvent (ηs), ηr=η/ηs.

“Ambient temperature” can be any temperature including and between 20° C. and 30° C.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

The “melting temperature,” Tm, of a polymer is the temperature at which an endothermal peak is observed in a DSC measurement, and where at least some of the crystallites begin to become disordered. The measured melting temperature may occur over a temperature range as the size of the crystallites, as well as presence of impurities and/or plasticizers, impacts the measured melting temperature of a polymer.

As used herein, a reference to the crystallinity of a polymer refers to the crystallinity as determined by standard DSC techniques.

As used herein, a “polymer” refers to a molecule comprised of, actually or conceptually, repeating “constitutional units.” The constitutional units derive from the reaction of monomers. As a non-limiting example, ethylene (CH₂═CH₂) is a monomer that can be polymerized to form polyethylene, CH₃CH₂(CH₂CH₂)ₙCH₂CH₃ (where n is an integer), wherein the constitutional unit is —CH₂CH₂—, ethylene having lost the double bond as the result of the polymerization reaction. Although poly(ethylene) is formed by the polymerization of ethylene, it may be conceptually thought of being comprised of the —CH₂— repeating unit, and thus conceptually the polymer could be expressed by the formula CH₃(CH₂)ₘCH₃ where m is an integer, which would be equal to 2n+2 for the equivalent number of ethylene units reacted to form the polymer. A polymer may be derived from the polymerization of two or more different monomers and therefore may comprise two or more different constitutional units. Such polymers are referred to as “copolymers.” “Terpolymers” are a subset of “copolymers” in which there are three different constitutional units. The constitutional units themselves can be the product of the reactions of other compounds. Those skilled in the art, given a particular polymer, will readily recognize the constitutional units of that polymer and will equally readily recognize the structure of the monomer from which the constitutional units derive. Polymers may be straight or branched chain, star-like or dendritic, or one polymer may be attached (grafted) onto another. Polymers may have a random disposition of constitutional units along the chain, the constitutional units may be present as discrete blocks, or constitutional units may be so disposed as to form gradients of concentration along the polymer chain. Polymers may be cross-linked to form a network.

As used herein, a polymer has a chain length of 50 constitutional units or more, and those compounds with a chain length of fewer than 50 constitutional units are referred to as “oligomers.” As used to differentiate between oligomers and polymers herein, the constitutional unit will be the smallest unique repeating unit. For example, for poly(lactide) the constitutional unit would be

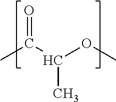

even though the polymer may be formed by the reaction of the cyclical dimer, lactide,

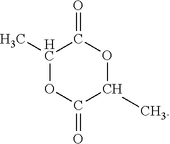

Similarly, for poly(ethylene) the constitutional unit used to count the “number” of constitutional units would be —CH₂— units, even though conventionally the constitutional unit is stated to be —CH₂CH₂— because it is always derived from the reaction of ethylene.

“Stress” refers to force per unit area, as in the force acting through a small area within a plane. Stress can be divided into components, normal and parallel to the plane, called normal stress and shear stress, respectively. True stress denotes the stress where force and area are measured at the same time. Conventional or engineering stress, as applied to tension and compression tests, is force divided by the original gauge length.

“Strength” refers to the maximum stress along an axis which a material will withstand prior to fracture. The ultimate strength is calculated from the maximum load applied during the test divided by the original cross-sectional area.

“Radial strength” of a stent is defined as the pressure at which a stent experiences irrecoverable deformation. The loss of radial strength is followed by a gradual decline of mechanical integrity.

“Modulus” may be defined as the ratio of a component of stress or force per unit area applied to a material divided by the strain along an axis of applied force that results from the applied force. The modulus is the initial slope of a stress-strain curve, and therefore, determined by the linear hookean region of the curve. For example, a material has a tensile, a compressive, and a shear modulus.

“Strain” refers to the amount of elongation or compression that occurs in a material at a given stress or load, or in other words, the amount of deformation.

“Elongation” may be defined as the increase in length in a material which occurs when subjected to stress. It is typically expressed as a percentage of the original length.

“Toughness” is the amount of energy absorbed prior to fracture, or equivalently, the amount of work required to fracture a material. One measure of toughness is the area under a stress-strain curve from zero strain to the strain at fracture. The units of toughness in this case are in energy per unit volume of material. See, e.g., L. H. Van Vlack, “Elements of Materials Science and Engineering,” pp. 270-271, Addison-Wesley (Reading, Pa., 1989).

As used herein, a “drug” refers to a substance that, when administered in a therapeutically effective amount to a patient suffering from a disease or condition, has a therapeutic beneficial effect on the health and well-being of the patient. A therapeutic beneficial effect on the health and well-being of a patient includes, but it not limited to at least one of the following: (1) curing the disease or condition; (2) slowing the progress of the disease or condition; (3) causing the disease or condition to retrogress; (4) alleviating one or more symptoms of the disease or condition.

As used herein, a “drug” also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to, at least one of the following: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; (3) preventing or delaying recurrence of the disease or condition after a course of treatment with a therapeutically effective amount of a substance, which may be the same as or different from the substance used in a prophylactically effective amount, has concluded.

As used herein, “drug” also refers to pharmaceutically acceptable, pharmacologically active salts, esters, amides, and the like, of those drugs specifically mentioned herein.

As used herein, a material that is described as “disposed over” an indicated substrate refers to, e.g., a coating layer of the material deposited directly or indirectly over at least a portion of the surface of the substrate. Direct depositing means that the coating layer is applied directly to the surface of the substrate. Indirect depositing means that the coating layer is applied to an intervening layer that has been deposited directly or indirectly over the substrate. A coating layer is supported by a surface of the substrate, whether the coating layer is deposited directly, or indirectly, onto the surface of the substrate. The terms “layer” and “coating layer” will be used interchangeably herein. A “layer” or “coating layer” of a given material is a region of that material whose thickness is small compared to both its length and width (e.g., the length and width dimensions may both be at least 5, 10, 20, 50, 100 or more times the thickness dimension in some embodiments). As used herein a layer need not be planar, for example, taking on the contours of an underlying substrate. Coating layers can be discontinuous. As used herein, the term “coating” refers to one or more layers deposited on a substrate. A coating layer may cover all of the substrate or a portion of the substrate, for example a portion of a medical device surface. A coating layer does not provide a significant fraction of the mechanical support for the device. In some embodiments, the layers differ from one another in the type of materials in the layer, the proportions of materials in the layer, or both. In some embodiments, a layer may have a concentration gradient of the components. One of skill in the art will be able to differentiate different coating layers or regions from each other based on the disclosure herein.

As used herein, “above” a surface or layer is defined as further from the substrate measured along an axis normal to a surface, or over a surface or layer, but not necessarily in contact with the surface or layer.

As used herein, “below” a surface or layer is defined as closer to the substrate measured along an axis normal to a surface, or under a surface or layer, but not necessarily in contact with the surface or layer.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention. Moreover, although individual aspects or features may have been presented with respect to one embodiment, a recitation of an aspect for one embodiment, or the recitation of an aspect in general, is intended to disclose its use in all embodiments in which that aspect or feature can be incorporated without undue experimentation. Also, embodiments of the present invention specifically encompass embodiments resulting from treating any dependent claim which follows as alternatively written in a multiple dependent form from all prior claims which possess all antecedents referenced in such dependent claim (e.g. each claim depending directly from claim 1 should be alternatively taken as depending from any previous claims).

What is claimed is:

1. A method of making a stent body for supporting a vascular lumen, comprising
immersing a cylindrical member in a solution comprising a bioabsorbable polymer dissolved in a fluid, wherein the bioabsorbable polymer has an inherent viscosity of at least 3.3 dl/g, has a number average molecular weight greater than 250,000 g/mole as measured by GPC using polystyrene standards, or both, and the solution further comprises an additive dissolved, dispersed, or a combination of dissolved and dispersed in the solution;
removing the member from the solution, wherein a portion of the solution remains on the surface of the member upon removal from the solution;
removing solvent from the solution remaining on the member to form a tubular layer of the bioabsorbable polymer and the additive on the member;
optionally, repeating on one or more occasions, the immersion operation, removal from the solution operation, and removal of the solvent operation to form a final tubular layer of bioabsorbable polymer and the additive on the member of a desired thickness; and
forming a stent body from the final tubular layer;
wherein the bioabsorbable polymer is poly(L-lactide), a copolymer with L-lactide or L-lactic acid as a constituent monomer, or a combination thereof; and
wherein at least one of the following conditions applies:
(a) the additive is the or at least one constituent monomer of the bioabsorbable polymer, and the additive is present at a weight ratio of the additive to the total of the additive and the polymer of about 0.002 to about 0.05;
(b) the additive is an oligomer of the or at least one constituent monomer of the bioabsorbable polymer, and the additive is present at a weight ratio of the additive to the total of the additive and the polymer of about 0.02 to about 0.25;
(c) the additive is a fatty acid at a weight ratio of the additive to the total of the additive and the polymer of about 0.002 to about 0.03;
(d) the additive is a fatty acid ester at a weight ratio of the additive to the total of the additive and the polymer of about 0.002 to about 0.03;
(e) the additive is an unsaturated fatty acid at a weight ratio of the additive to the total of the additive and the polymer of about 0.002 to about 0.03;
(f) the additive is an unsaturated fatty acid ester at a weight ratio of the additive to the total of the additive and the polymer of about 0.002 to about 0.03;
(g) the additive is a hydroxy acid;
(h) the additive is an ester of a hydroxy acid, wherein if the or at least one constituent monomer of the bioabsorbable polymer is a hydroxy acid or a hydroxy acid ester, the additive is a different hydroxy acid ester;
(i) the additive is a dicarboxylic acid;
(j) the additive is an ester of a dicarboxylic acid;
(k) the additive is an anhydride;
(l) the additive is an acid or ester of an acid selected from the group consisting of citric acid, ascorbic acid, erythorbic acid, thiodipropionic acid, cholic acid, desoxycholic acid, glycocholic acid, taurocholic acid, aspartic acid, tartaric acid, glutamic acid, and combinations thereof;
(m) the additive is a metal ion selected from the group consisting of zinc, aluminum, tin, magnesium, calcium, sodium, and iron;
(n) the additive is a hygroscopic additive.

2. The method claim 1, wherein the member is removed from the solution in less than 30 seconds.

3. The method claim 1, wherein the member is immersed with its cylindrical axis perpendicular to the surface of the solution.

4. The method claim 1, wherein the member is rotated 180° prior to repetition of the immersion step.

5. The method of claim 1, wherein the member is rotated while it is removed from the solution.

6. The method claim 1, further comprising radially expanding the final tubular layer and forming the stent body from the expanded tube.

7. The method claim 1, wherein condition (a), (b), or a combination thereof apply, and wherein the additive is selected from the group consisting of D,L-lactide, D,D-lactide, L,L-lactide, meso-lactide, glycolide, ε-caprolactone, trimethylene carbonate, p-dioxanone, γ-valeroactone, γ-undecalactone, β-methyl-δ-valerolactone, anhydrides, orthocarbonates, phosphazenes, orthoesters, amino acids, and combinations thereof.

8. The method claim 1, wherein condition (c), (d), or a combination thereof apply, and wherein the fatty acid, the fatty acid of the fatty acid ester, or a combination thereof is selected from the group consisting acetic acid, propanoic acid, butyric acid, caprylic acid, caproic acid, capric acid, lauric acid, myristic acid, palmitic acid, stearic acid, arachidic acid, behenic acid, lignoceric acid, cerotic acid, and combinations thereof.

9. The method claim 1, wherein condition (e), (f), or a combination thereof apply, and wherein the unsaturated fatty acid, the unsaturated fatty acid of the unsaturated fatty acid ester, or a combination thereof, is selected from the group consisting of myristoleic acid, palmitoleic acid, spienic acid, oleic acid, elaidic acid, vaccenic acid, linoleic acid, linoelaidic acid, alpha-linolenic acid, arachidonic acid, eicosapentaenoic acid, erucic acid, and docosahexaenoic acid, and combinations thereof.

10. The method claim 1, wherein condition (g), (h), or a combination thereof apply, and wherein the hydroxy acids are selected from the group consisting of L-lactic acid, D-lactic acid, glycolic acid, 2-hydroxybutyric acid, 3-hydroxybutyric acid, 2-hydroxyvaleric acid, 3-hydroxybutyric acid, 4-hydroxybutyric acid, 3-hydroxyvaleric acid, 4-hydroxyvaleric acid, 5-hydroxyvaleric acid, dimethylglycolic acid, β-hydroxypropanic acid, α-hydroxybutyric acid, α-hydroxycaproic acid, β-hydroxycaproic acid, γ-hydroxycaproic acid, δ-hydroxycaproic acid, δ-hydroxymethylcaproic acid, ε-hydroxycaproic acid, ε-hydroxymethylcaproic acid, citric acid, tartaric acid, and combinations thereof.

11. The method claim 1, wherein condition (i), (j), or a combination thereof applies, and the dicarboxylic acid, the dicarboxylic acid of the ester, or a combination thereof, is selected from the group consisting of oxalic acid, malonic acid, succinic acid, glutaric acid, adipic acid, pimelic acid, suberic acid, azelaic acid, sebacic acid, orthophthalic acid, isophthalic acid, terephthalic acid and combinations thereof.

12. The method claim 1, wherein condition (k) applies, and the anhydride is selected from the group consisting of succinic anhydride, glutaric anhydride, maleic anhydride, acetic anhydride, propanoic anhydride, butyric anhydride, valeric anhydride, caproic anhydride, heptanoic anhydride, phthalic anhydride, and benzoic anhydride, and combinations thereof.

13. The method claim 1, wherein condition (l) applies.

14. The method claim 1, wherein condition (m) applies.

15. The method of claim 1, wherein condition (n) applies, and the hygroscopic additive is selected from the group consisting of sodium phosphate, sodium biphosphate, sodium pyrophosphate, potassium phosphate, sodium carbonate, sodium bicarbonate, potassium carbonate, sodium sulfate, magnesium sulfate, sodium chloride, potassium chloride, calcium ascorbate, calcium propionate, calcium sorbate, calcium carbonate, calcium citrate, calcium glycerophosphate, calcium oxide, calcium pantothenate, calcium phosphate, calcium pyrophosphate, calcium sulfate, calcium chloride, calcium gluconate, calcium hydroxide, calcium lactate, calcium oxide, magnesium chloride, methyl cellulose, ethyl cellulose, sodium carboxymethylcellulose, and cellulose acetate, and combinations thereof.

16. The method of claim 1, wherein condition (n) applies, wherein the hygroscopic additive is present at a weight ratio of the additive to the total of the additive and the polymer of about 0.002 to about 0.05; and wherein the additive is propylene glycol, glycerol, or a combination thereof.

17. A method of making a stent body for supporting a vascular lumen, comprising
immersing a cylindrical member in a solution comprising a bioabsorbable polymer dissolved in a solvent, wherein the bioabsorbable polymer has an inherent viscosity of at least 3.3 dl/g, has a number average molecular weight greater than 250,000 g/mole as measured by GPC using polystyrene standards, or both;
removing the member from the solution, wherein a portion of the solution remains on the surface of the member upon removal from the solution;
removing solvent from the solution remaining on the member to form a tubular layer of the bioabsorbable polymer on the member;
optionally, repeating on one or more occasions the immersion operation, removal from the solution operation, and removal of the solvent operation to form a final tubular layer of bioabsorbable polymer on the member of a desired thickness; and
forming a stent body from the final tubular layer;
wherein at least one of the following conditions applies:
(a) the polydispersity of the bioabsorbable polymer is at least 4 or greater than 4;
(b) wherein the bioabsorbable polymer is poly(L-lactide), a copolymer where one constituent monomer is L-lactide, or a combination thereof; and wherein the solution further comprises a second bioabsorbable polymer, the second bioabsorbable polymer being poly(glycolide), a copolymer where one constituent monomer is glycolide, poly(D,L-lactide), a copolymer where one constituent monomer is D,L-lactide, polydioxanone, poly(4-hydroxybutyrate), poly(trimethylene carbonate), a copolymer where at least one constituent monomer is polydioxanone, poly(4-hydroxybutyrate), or poly(trimethylene carbonate), or a combination thereof.

18. The method of claim 17, wherein condition (a) applies.

19. The method of claim 17, wherein condition (b) applies.

20. The method of claim 19, wherein the second bioabsorbable polymer is of a number average molecular weight of not more than one fifth of the number average molecular weight of the first polymer.

21. A polymer scaffold comprising
a device body made of a bioabsorbable polymer,
and optionally, an additive;
wherein at least one of the following conditions applies:
(a) the polydispersity of the bioabsorbable polymer is at least 4 or greater than 4;
(b) wherein the bioabsorbable polymer is poly(L-lactide), a polymer of which at least one constituent monomer of the polymer is L-lactide, or a combination thereof; and wherein a second bioabsorbable polymer is blended with the bioabsorbable polymer, the second bioabsorbable polymer being poly(glycolide), a copolymer where one constituent monomer is glycolide, poly(D,L-lactide), a copolymer where one constituent monomer is D,L-lactide, polydioxanone, poly(4-hydroxybutyrate), poly(trimethylene carbonate), a copolymer where at least one constituent monomer is polydioxanone, poly(4-hydroxybutyrate), or poly(trimethylene carbonate), or a combination thereof;
(c) an additive is present, and if the additive is the or at least one constituent monomer of the bioabsorbable polymer, the additive is present at a weight ratio of the additive to the total of the additive and the polymer of about 0.002 to about 0.05; if the additive is an oligomer of the or at least one constituent monomer of the bioabsorbable polymer, the additive is present at a weight ratio of the additive to the total of the additive and the polymer of about 0.02 to about 0.25; if the additive is a fatty acid, a fatty acid ester, an unsaturated fatty acid, an unsaturated fatty acid ester, the additive is present at a weight ratio of the additive to the total of the additive and the polymer of about 0.002 to about 0.03.

22. The scaffold of claim 21, wherein condition (a) applies.

23. The scaffold of claim 21, wherein condition (b) applies, and wherein the second polymer is of a number average molecular weight of not more than one fifth of the number average molecular weight of the first polymer.

24. The scaffold of claim 21, wherein condition (c) applies, and the additive is a member of at least one of the following groups:
(a) the constituent monomer(s) of the bioabsorbable polymer;
(b) oligomers formed from the or at least one constituent monomer of the bioabsorbable polymer;
(c) fatty acids;
(d) fatty acid esters;
(e) unsaturated fatty acids;
(f) unsaturated fatty acid esters;
(g) hydroxy acids;
(h) esters of hydroxy acids, wherein if the or at least one constituent monomer of the bioabsorbable polymer is a hydroxy acid or hydroxyacid diester including cyclic diesters, the additive is a different hydroxy acid;
(i) dicarboxylic acids;
(j) esters of dicarboxylic acids;
(k) anhydrides;
(l) acids, esters of an acid, and combinations thereof, wherein the acid is selected from the group consisting of an acid or ester of an acid selected from the group consisting of citric acid, ascorbic acid, erythorbic acid, thiodipropionic acid, cholic acid, desoxycholic acid, glycocholic acid, taurocholic acid, aspartic acid, tartaric acid, glutamic acid, and combinations thereof;
(m) metal ions selected from the group consisting of zinc, iron, tin, magnesium, calcium, sodium and aluminum;
(n) the additive is a hygroscopic additive.
